(19) United States
(12) Patent Application Publication
(10) Pub. No.: US 2026/0248767 A1
(43) Pub. Date: Aug. 27, 2026

(54) METHODS FOR INDUCING FERROPTOSIS IN CANCER

(71) Applicant: Board of Regents, The University of Texas System, Austin, TX (US)

(72) Inventors: Boyi Gan, Austin, TX (US); Yuelong Yan, Austin, TX (US)

(21) Appl. No.: 19/163,744

(22) PCT Filed: Mar. 12, 2024
(86) PCT No.: PCT/US2024/019602

§ 371 Date: Sep. 9, 2025

Related U.S. Application Data
(60) Provisional application No. 63/452,358, filed on Mar. 15, 2023.

(51) Int. Cl. A61K 31/4162; A61K 31/135; A61K 31/18; A61K 31/198; A61K 31/357; A61K 31/381; A61K 31/437; A61K 31/495; A61K 31/496; A61K 31/519; A61K 33/04; A61P 35/00

(52) U.S. Cl. CPC A61K 31/4162; A61K 31/135; A61K 31/18; A61K 31/198; A61K 31/357; A61K 31/381; A61K 31/437; A61K 31/495; A61K 31/496; A61K 31/519; A61K 33/04; A61P 35/00

(57) ABSTRACT

The present disclosure provides methods for treating cancer using ferroptosis inducers in combination with one-carbon metabolism inhibitors. The combination of ferroptosis inducers and one-carbon metabolism inhibitors induce greater ferroptosis than treatment with either alone. In certain embodiments, the one-carbon metabolism inhibitors are serine hydroxymethyltransferase (SHMT) inhibitors and/or methylenetetrahydrofolate dehydrogenase (MTHFD) inhibitors. By way of example, the ferroptosis inducers are GPX4 inhibitors.

### CROSS-REFERENCE TO RELATED APPLICATIONS

This application claims benefit of priority under 35 U.S.C. § 119 (e) of U.S. Provisional Application No. 63/452,358, filed Mar. 15, 2023. The prior application is considered part of and is herein incorporated by reference in this application in its entirety.

### STATEMENT OF GOVERNMENT RIGHTS

This invention was made with government support under Grant No. RO1CA181196 awarded by the National Institutes of Health. The government has certain rights in the invention.

### INCORPORATION OF SEQUENCE LISTING

The material in the accompanying sequence listing is hereby incorporated by reference into this application. The accompanying sequence listing xml file, name MDA1130-1WO_SL.xml was created on Feb. 27, 2024, and is 10 kb.

### BACKGROUND OF THE INVENTION

### Field of the Invention

The present disclosure is related generally to methods of treating cancer and more specifically to using GPX4 inhibitors in combination with SHMT inhibitors to induce ferroptosis and treat cancer.

### Background Information

A hallmark of cancer is the development of resistance to apoptosis, often through genetic loss of the molecular machinery involved in programmed cell death. Furthermore, resistance to chemotherapeutics and molecular targeted therapies are major challenges in oncology. As a result, harnessing an understanding of non-apoptotic cell death pathways, such as ferroptosis, has substantial therapeutic potential for patients. Ferroptosis is an iron-dependent, non-apoptotic form of regulated cell death characterized by aberrant lipid membrane peroxidation. Given that dysregulated iron metabolism and iron accumulation have been frequently observed across both solid tumors and hematological malignancies, selectively inducing ferroptosis is an attractive potential anti-cancer strategy with broad clinical implications.

There has been great interest in targeting one carbon metabolism and ferroptosis in cancer therapy. However, therapeutic efficacy by single treatments is likely limited. Therefore, there is a need to identify combination therapies targeting these pathways. SHMT1/2 are enzymes involved in one-carbon metabolism. GPX4 is an enzyme involved in suppressing ferroptosis (an iron-dependent cell death induced by lipid peroxidation).

### SUMMARY OF THE INVENTION

The present disclosure is based on the seminal discovery that serine hydroxymethyltransferase (SHMT) inhibition sensitizes cancer cells to glutathione peroxidase 4 (GPX4) inhibition-induced ferroptosis showing that the combination of an SHMT inhibitor and a GPX4 inhibitor is an effective combination therapeutic strategy for treating cancer.

In one embodiment, the present disclosure provides a method of treating cancer in a subject that includes administering to the subject: a) one or more one-carbon metabolism inhibitors; and b) one or more ferroptosis inducers, wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby treating cancer in the subject.

In one embodiment, the present disclosure provides a method of treating cancer in a subject that includes administering to the subject: a) one or more one-carbon metabolism inhibitors; and b) one or more ferroptosis inducers, wherein the ferroptosis induced in the tumor is greater than the ferroptosis induced when the tumor is exposed to one or more ferroptosis inducers or one or more one-carbon metabolism inhibitors alone, and wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby enhancing ferroptosis in the tumor.

In another embodiment, the present disclosure provides a method of sensitizing cancer cells to ferroptosis that includes exposing the cancer cells to one or more one-carbon metabolism inhibitors, wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby sensitizing the cancer cells to ferroptosis.

In one aspect, ferroptosis is induced by administering one or more ferroptosis inducers. For example, in one aspect the ferroptosis inducer is a glutathione peroxidase 4 (GPX4) inhibitor. In one aspect, the SHMT inhibitor is an SHMT1 inhibitor, an SHMT2 inhibitor or an SHTM1/SHMT2 inhibitor. In one aspect, the MTHFD inhibitor is a MTHFD1 inhibitor, MTHFD2 inhibitor, an MTHFD1L inhibitor or a combination thereof.

In one aspect, the one or more one-carbon metabolism inhibitors and the one or more ferroptosis inducers are administered at the same time.

In one aspect, the one or more one-carbon metabolism inhibitors and the one or more ferroptosis inducers are administered sequentially.

In one aspect, at least one SHMT inhibitor is SHIN2, AGF347, SHIN1, sertraline, Compound 2.12, Compound 2.2, sulfite, S-sulfocysteine, or a combination thereof.

In one aspect, at least one GPX4 inhibitors is RSL3, FIN56, FINO₂, ML-210, M-162, JKE-1674 or a combination thereof.

In one aspect, administering the one or more one-carbon metabolism inhibitors and the one or more ferroptosis inducers results in an increase in ferroptosis.

In one aspect, the cancer is leukemia, breast cancer, kidney cancer, brain cancer, non-small cell lung cancer, prostate cancer, colon cancer, cervical cancer, or fibrosarcoma.

In one aspect, an effective dosage of at least one of the SHMT inhibitor, the MTHFD inhibitor, and the GPX4 inhibitor is administered to a subject in need thereof. In another aspect, the effective dosage of the SHMT inhibitor is about 0.0025 to about 1500 mg/kg, the effective dosage of the MTHFD inhibitor is about 0.0025 to about 1500 mg/kg, and the effective dosage of the GPX4 inhibitor is about 0.0025 to about 1500 mg/kg.

In one embodiment, the present disclosure provides a pharmaceutical composition that includes a) one or more one-carbon metabolism inhibitors; b) one or more ferroptosis inducers; and c) a pharmaceutically acceptable carrier. In one aspect, at least one one-carbon metabolism inhibitor is a serine hydroxymethyltransferase (SHMT) inhibitor, a methylenetetrahydrofolate dehydrogenase (MTHFD) inhibitor or a combination thereof. In one aspect, the SHMT inhibitor is an SHMT1 inhibitor, an SHMT2 inhibitor or an SHTM1/SHMT2 inhibitor. In one aspect, the SHMT inhibitor is SHIN2, AGF347, SHIN1, sertraline, Compound 2.12, Compound 2.2, sulfite, S-sulfocysteine, or a combination thereof. In one aspect, the MTHFD inhibitor is a MTHFD1 inhibitor, MTHFD2 inhibitor, an MTHFD1L inhibitor or a combination thereof. In one aspect, at least one ferroptosis inducer is a glutathione peroxidase 4 (GPX4) inhibitor. In one aspect, the GPX4 inhibitor is RSL3, FIN56, ML-210, M-162, JKE-1674 or a combination thereof. In another aspect, a unit dose of the SHMT inhibitor when present is about 0.01 mg to about 1 g, a unit dose of the MTHFD inhibitor when present is about 0.01 mg to about 1 g, and a unit dose of the GPX4 inhibitor when present is about 0.01 mg to about 1 g.

### BRIEF DESCRIPTION OF THE DRAWINGS

FIG. 1A is a graph showing the effect of sulfite, sulfite plus RSL3 and sulfite plus RSL3 and Ferr-1 on cell death in multiple cell lines.

FIG. 1B is a graph showing that sulfite reduces cell viability when combined with different concentrations of RSL3.

FIG. 1C is a graph illustrating cell death observed with combination of sulfite with ferroptosis inducers ML162 and ML162+Fer-1.

FIG. 1D is a graph illustrating cell death observed with combination of sulfite with ferroptosis inducer ML-162.

FIG. 1E is a graph illustrating cell death observed with combination of sulfite with ferroptosis inducer ML-210.

FIG. 1F is a graph illustrating cell death observed with combination of sulfite with ferroptosis inducer ML-210.

FIG. 1G is a graph illustrating cell death observed with combination of sulfite with ferroptosis inducer JKE-1674.

FIG. 1H is a graph illustrating cell death observed with combination of sulfite with ferroptosis inducer JKE-1674.

FIG. 1I is a graph illustrating cell death observed with combination of sulfite with ferroptosis inducer FIN56.

FIG. 1J is a graph illustrating cell death observed with combination of sulfite with ferroptosis inducer FIN56.

FIG. 1K and FIG. 1L are graphs showing relative lipid peroxidation and relative mitochondrial lipid peroxidation in RSL3 and/or sulfite treated cells.

FIG. 1M and FIG. 1N are graphs showing relative lipid peroxidation and relative mitochondrial lipid peroxidation in FIN56 and/or sulfite treated cells.

FIG. 1O and FIG. 1P are graphs showing cell death under RSL3 and/or sulfite treatment with mitochondria specific lipid peroxidation neutralizer.

FIG. 2A is a is a graph showing how cysteine is metabolized to produce sulfite.

FIG. 2B is a graph showing effect of sulfite, sulfate, and thiosulfate on cell viability.

FIG. 2C is a graph showing cell death in cell treated with RSL3 and sulfite or thiosulfate.

FIG. 2D is a graph showing cell death in cell treated with RSL3 and sulfite or sulfate.

FIG. 2E and FIG. 2F are graphs showing cell death with different cell death inhibitors.

FIG. 2G is an image showing SUOX protein levels in SUOX knock out cells.

FIG. 2H is a graph showing the effect of RSL3 and sulfite treatment on cell death of SUOX knockout cells.

FIG. 2I is a graph showing the cell death observed with RSL3 or RSL3 plus Fer-1 in SUOX knockout cells.

FIG. 2J is a graph showing the relative lipid peroxidation level.

FIG. 2K is a graph showing the relative mitochondrial lipid peroxidation level in RSL3 treated SUOX knockout cells.

FIG. 2L and FIG. 2M are graphs showing cell death observed with RSL3 plus with mitochondria specific lipid peroxidation neutralizer in SUOX knock out cells.

FIGS. 3A-3P. FIG. 3A is an image showing metabolic analysis results in sulfite treated cells. FIG. 3B is an image showing the pathway enrichment of changed metabolites induced by sulfite treatment. Sulfite treatment associated changes in 6-phospho-D-Gluconate (see graph of FIG. 3C), ribose phosphate (see graph of FIG. 3D), D-Glyceraldehyde 3-phosphate (see graph of FIG. 3E), D-sedoheptulose-1/7 phosphate (see graph of FIG. 3F), NDAP+/NADPH ratio (see graph in FIG. 3G), oxPPP shunting (see graph of FIG. 3H) also shown in the corresponding figures. Cell death observed with RSL3 in combination with 6-AN or EA are shown in graphs of FIG. 3I and FIG. 3J. FIG. 3K is an image showing protein levels of IDH1, ME1, MTHFD2. FIG. 3L is a graph showing NADP+/NADPH ratio in IDH1, ME1, or MTHFD2 knockdown cells. FIG. 3M is an image that shows how serine is metabolized through one-carbon metabolism pathway. The total dTTP, M+1dTTP and M+2 dTT in the presence or absence of sulfite are shown in graphs of FIG. 3N, FIG. 30, and FIG. 3P.

FIGS. 4A-4M. FIG. 4A is an image of a reaction showing cystine react with sulfite to generate S-sulfocysteine. FIG. 4B is a graph showing levels of S-sulfocysteine in sulfite treated samples. FIG. 4C is a graph showing cell death in SSC plus RSL3 treated cells. FIG. 4D is a graph showing cell viability in cells treated with SSC and multiple concentrations of RSL3. FIG. 4E, FIG. 4F, FIG. 4G, FIG. 4H are graphs showing cell death in SSC plus ML-162, ML-210, JKE-1674, and FIN56 treated cells respectively. FIG. 4I and FIG. 4J are graphs showing lipid peroxidation levels observed in RSL3 and S-sulfocysteine or FIN56 and S-sulfocysteine respectively. FIG. 4K and FIG. 4L are graphs showing mitochondrial lipid peroxidation levels observed in RSL3 and S-sulfocysteine or FIN56 and S-sulfocysteine respectively. FIG. 4M are graphs showing cell death induced by sulfite plus RSL3 with either normal or low concentration of cystine.

FIGS. 5A-5Z. FIG. 5A is an image showing SHMT2 protein levels in SHMT2 knockout cells. FIG. 5B is a graph showing cell viability in SHMT2 knockout cells treated with multiple concentrations of RSL3. FIG. 5C is a graph showing cell death in SHMT2 knockout cells treated with RSL3. FIG. 5D is a graph showing lipid peroxidation levels observed in RSL3 treated SHMT2 knock out cells. FIG. 5E is a graph showing mitochondrial lipid peroxidation levels observed in RSL3 treated SHMT2 knock out cells. FIG. 5F plus FIG. 5G, FIG. 5I are graphs showing cell death in SHMT2 knockout cells treated with ML-162, JKE-1674, and FIN56 respectively. FIG. 5H is a graph showing cell viability in SHMT2 knock out cells treated with JKE-1674. FIG. 5J and is a graph showing cell death in SHMT2 knock out cells treated with RLS3 or RSL3 plus sulfite treatment. FIG. 5K is a graph showing cell viability in SHMT2 knock out cells treated with sulfite plus multiple concentrations of RSL3. FIG. 5L, FIG. 5Q, FIG. 5V are graphs showing that SHIN1, SHIN2, AGF347 respectively increase cell death in combination with RSL3. FIG. 5M, FIG. 5R, FIG. 5W are graphs showing that SHIN1, SHIN2, AGF347 respectively increase lipid peroxidation in combination with RSL3. FIG. 5N, FIG. 5S, FIG. 5X are graphs showing that SHIN1, SHIN2, AGF347 respectively increase mitochondrial lipid peroxidation in combination with RSL3. FIG. 5O, FIG. 5T, FIG. 5Y are graphs showing that SHIN1, SHIN2, AGF347 respectively increase cell death in combination with ML-162. FIG. 5P, FIG. 5U, FIG. 5Z are graphs showing that SHIN1, SHIN2, AGF347 respectively increase cell death in combination with FIN56.

FIGS. 6A-6F. FIG. 6A is an image showing MTHFD2 protein levels in MTHFD2 knockout cells. FIG. 6B is a graph showing cell death in RSL3 treated MTHFD2 knockout cells. FIG. 6C is a graph showing cell death in ML-162 treated MTHFD2 knockout cells. FIG. 6D is a graph showing cell death in JKE-1674 treated MTHFD2 knockout cells. FIG. 6E is a graph showing NADP+/NADPH ratio in SHMT2 knockout cells. FIG. 6F is a graph showing NADP+/NADPH ratio in SHIN1 or SHIN2 treated cells.

FIGS. 7A-7I. FIG. 7A is a graph showing cell death in multiple cancer cells treated with SHIN1 plus RSL3. FIG. 7B is a graph showing cell death in multiple cancer cells treated with SHIN2 plus RSL3. FIG. 7C, FIG. 7D, FIG. 7E are graphs showing cell death, lipid peroxidation and mitochondrial lipid peroxidation in RSL3 plus sertraline treated cells. FIG. 7F is a graph showing cell death in ML-162 and sertraline treated cells. FIG. 7G is a graph showing cell death in JKE-1674 and SHIN2 treated cells. FIG. 7H is a graph showing cell death in JKE-1674 plus sertraline treated cells. FIG. 7I is a graph showing cell death in SHIN1, SHIN2, AGF347, and sertraline treated cells which were also treated with various concentrations of RSL3.

FIG. 8A is an image showing sulfite treatment inhibits the tetramer formation of endo SHMT2 protein. FIG. 8B is an image showing the exogenous expressed SHMT2 can form tetramer. FIG. 8C is an image showing sulfite can also inhibit the tetramer formation of exogenous SHMT2 protein.

FIG. 9 is an image showing SHMT2 and MTHFD2 protein levels in sulfite treated cells.

FIGS. 10A-10J. FIG. 10A, FIG. 10B, and FIG. 10C are graphs, FIG. 10D is an image, FIG. 10E is a graph, FIG. 10F is an image, FIG. 10G is a graph, FIG. 10H is an image, and FIG. 10I and FIG. 10J are graphs showing that sulfite sensitization effect did not depend on single known ferroptosis regulator.

FIG. 11A is a graph illustrating cell death of HT-1080 cells treated with SHIN2 for 24 hrs.

FIG. 11B is a graph illustrating cell death of HT-1080 cells treated with steraline for 24 hrs.

FIG. 11C is a graph illustrating tumor volumes of HT-1080 derived xenograft with treated with PBS, sertraline, JKE-1674, JKE+sertraline, or JKE+sertraline+Lip-1.

FIG. 11D is a graph illustrating synergy score of the combination of sertraline plus JKE-1674 treatment in HT-1080 derived xenograft.

FIG. 11E is a graph illustrating mice body weight of HT-1080 derived xenograft treated with PBS, sertraline, JKE-1674, JKE+sertraline, or JKE+Ser+Lip-1.

FIG. 11F is a set of images illustrating immunostaining of H&E, cleaved caspase-3, ki67 and 4-HNE from tumors treated with vehicle, sertraline, JKE-1674, JKE-1674+sertraline, or JKE-1674+sertraline+Lip-1.

FIG. 11G is a graph illustrating immunochemistry scoring of cleaved caspase-3 staining in tumors treated with vehicle, sertraline, JKE-1674, JKE+sertraline, and JKE+sertraline+Lip-1.

FIG. 11H is a graph illustrating immunochemistry scoring of KI-67 staining in tumors treated with vehicle, sertraline, JKE-1674, JKE+sertraline, and JKE+sertraline+Lip-1.

FIG. 11I is a graph illustrating immunochemistry scoring of 4-HNE staining in tumors treated with vehicle, sertraline, JKE-1674, JKE+sertraline, and JKE+sertraline+Lip-1.

FIG. 11J is a graph illustrating cell death of LKR-13 cells treated with DMSO, RSL3 or RSL3+Fer-1 for 24 hrs.

FIG. 11K is a graph illustrating cell death of LKR-13 cells treated with DMSO, JKE or JKE+Fer-1 for 24 hrs.

FIG. 11L is a graph illustrating tumor volume of LKR-13 derived xenograft treated with PBS, sertraline, JKE, JKE+sertraline, or JKE+sertraline+Lip-1.

FIG. 11M is a graph illustrating tumor weight of LKR-13 derived xenograft treated with PBS, sertraline, JKE, JKE+sertraline, or JKE+sertraline+Lip-1.

FIG. 11N is a graph illustrating synergy score for the combination of sertraline plus JKE-1674 treatment in LKR-13 derived xenograft.

FIG. 11O is a graph illustrating body weight of LKR-13 derived xenograft treated with PBS, sertraline, JKE, JKE+sertraline, or JKE+sertraline+Lip-1.

### DETAILED DESCRIPTION OF THE INVENTION

The present invention is based on the seminal discovery that GPX4 inhibitors in combination with SHMT inhibitors induce ferroptosis, inhibiting tumor growth and reducing tumor weight in a synergistic manner.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

Although the term “at least one” may often be used in the specification, claims and drawings, the terms “a”, “an”, “the”, “said”, etc. also signify “at least one” or “the at least one” in the specification, claims and drawings.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The presently described methods and compounds are useful for detecting, predicting, treating and/or monitoring cancer status in a subject. Any appropriate subject, such as a mammal can be assessed, monitored, and/or treated as described herein. Examples of some mammals that can be assessed, monitored, and/or treated as described herein include, without limitation, humans, primates such as monkeys, dogs, cats, horses, cows, pigs, sheep, mice, and rats. For example, a human having, or suspected of having, cancer can be assessed using a method described herein and, optionally, can be treated with one or more cancer treatments as described herein.

Ferroptosis is a form of non-apoptotic cell death induced by excessive lipid peroxidation. Cells have evolved at least two defense mechanisms to suppress ferroptosis. In the first, glutathione peroxidase 4 (GPX4) utilizes reduced glutathione (GSH) to detoxify lipid hydroperoxides and inhibit ferroptosis. Cancer cells rely on solute carrier family 7 member 11 (SLC7A11)-mediated cystine transport to obtain cysteine for GSH biosynthesis. The SLC7A11-GPX4 signaling axis represents a cellular defense system against ferroptosis, and inactivation of GPX4 or SLC7A11 by corresponding ferroptosis inducers induces ferroptosis in many cancer cells. In the second, ferroptosis suppressor protein 1 (FSP1; also called AIFM2) acts as another ferroptosis inhibitor that acts in parallel to GPX4 to suppress ferroptosis. Whether there exist additional cellular defense mechanisms against ferroptosis at other subcellular compartments remains unclear.

Ferroptosis has recently emerged as an important tumor suppression mechanism. Despite an emerging understanding of the role of ferroptosis in tumor suppression, there is an unmet need to identify the specific context for therapeutic targeting of ferroptosis and to develop therapeutic agents that sensitize cancer cells to ferroptosis and/or rational drug combination strategies.

There has been great interest in targeting one-carbon metabolism and ferroptosis in cancer therapy. However, therapeutic efficacy by single treatments is likely limited; therefore, there is an unmet need to identify novel rational combination therapies targeting these pathways. In the present disclosure, the inventors made the discovery that one-carbon metabolism inhibitors, e.g., SHMT inhibitors, sensitize cancer cells to ferroptosis induced by GPX4 inhibitors. Surprisingly, it is shown herein that one-carbon metabolism inhibitors, e.g., SHMT inhibitors alone did not induce ferroptosis or cell death. However, when SHMT inhibitors were combined with GPX4 inhibitors, an unexpected synergistic increase in cell death was observed. This combination therapy provides new effective combination therapy in cancer treatment.

Altered metabolism in cancer cells is important for tumor growth. One of the most notable aspects of this metabolic reprogramming lies in one-carbon metabolism. Cells require one-carbon units for nucleotide synthesis, methylation reactions, and for the generation of a broad range of biosynthetic reactions that occur in the cytoplasm and the mitochondria which maintain cellular homeostasis. These pathways catabolize different carbon sources to derive one-carbon (methyl) units to be utilized in cellular functions. Due to the specific manner in which one-carbon units are obtained and utilized, one-carbon metabolism serves as an integrative pathway, relating many nutrients to one another.

One-carbon units are utilized in two pathways: the folate cycle and the methionine cycle. In the folate cycle, folic acid is reduced by dihydrofolate reductase (DHFR) to the biologically active tetrahydrofolate (THF). In this reduced form, one-carbon units from serine and glycine are transferred by serine hydroxymethyltransferase (SHMT) and glycine decarboxylase (GLDC; of the glycine cleavage system [GCS]), respectively, onto THE, forming methyl-THF. Once methylated, THF undergoes a series of redox transformations by the multi-functional enzyme methylenetetrahydrofolate dehydrogenase (MTHFD1/2/1L), which has cytosolic and mitochondrial isoforms. In the methionine cycle, homocysteine is re-methylated using a one-carbon unit from methyl-THF to form methionine via methionine synthase (MS). Demethylation of S-adenosyl-methionine (SAM) yields S-adenosyl-homocysteine (SAH), which is then converted to homocysteine, completing the cycle. Enzymes in the one-carbon metabolism also feed into other metabolic reactions involving complex feedback mechanisms. Therefore, the effect of one enzyme does not necessarily predict the anticipate the effect of other downstream or parallel metabolic reactions.

In one embodiment, the methods of the disclosure utilize one-carbon metabolism inhibitors. In another embodiment, the one-carbon metabolism inhibitor is a serine hydroxymethyltransferase (SHMT) inhibitor, and/or a methylenetetrahydrofolate dehydrogenase inhibitor. In one embodiment, the methylenetetrahydrofolate dehydrogenase (MTHFD) inhibitor is a MTHFD1 inhibitor, a MTHFD2 inhibitor and/or a MTHFD1L inhibitor. In another aspect, the one-carbon metabolism inhibitor is a dihydrofolate reductase (DHFR) inhibitor, a glycine decarboxylase inhibitor, and/or a methionine synthase (MS) inhibitor. In one embodiment, the one-carbon metabolism inhibitor is a cytosolic one-carbon metabolism inhibitor and/or a mitochondrial one-carbon metabolism inhibitor.

Serine hydroxymethyltransferase (SHMT) is an enzyme which plays an important role in cellular one-carbon pathways by catalyzing the reversible conversions of L-serine to glycine. In addition, SHMT catalyzes the conversion of tetrahydrofolate to 5,10-methylenetetrahydrofolate (e.g., SHMTs catalyze a reversible reaction converting serine to glycine, with concurrent methylenetetrahydrofolate (meTHF) generation). In one embodiment, the methods of the disclosure utilize SHMT inhibitors. In one embodiment, SHMT is SHMT1 in the cytosol and/or SHMT2 in the mitochondria.

Modulation of one-carbon metabolism is suitable for therapeutic intervention, such as in cancer. The present disclosure provides SHMT inhibitors. In one embodiment, the SHMT inhibitor is an SHMT1 inhibitor, an SHMT2 inhibitor and/or an inhibitor of both SHMT1 and SHMT2 (referred to herein as an SHTM1/SHMT2 inhibitor). In one embodiment, the disclosure provides compounds and methods to modulate (e.g., inhibit) serine flux and/or the mitochondrial folate pathway by inhibiting SHMT2 (e.g., SHMT2 inhibitors, providing compounds capable of inhibiting SHMT2). Such inhibitors optionally also inhibit SHMT1. Similarly, in one embodiment, the disclosure provides compounds and methods to modulate (e.g., inhibit) generation of NADPH by inhibiting SHMT2 (e.g., using inhibitors of SHMT2; providing compounds capable of inhibiting SHMT2). Such inhibitors optionally also inhibit SHMT1. In one embodiment, such SHMT inhibitors are selective inhibitors for SHMT enzymes (e.g., the compounds show selectivity for SHMT enzymes over DHFR and/or TS and/or MTHFD2). In one embodiment, suitable inhibitors of mammalian SHMT2 and/or SHMT1 do not substantially inhibit the activity of DHFR and/or TS and/or MTHFD2.

In one embodiment, the SHMT inhibitor is Compound 2.12 or Compound 2.2. In one

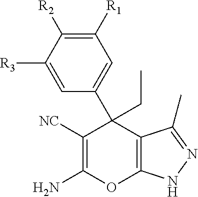

embodiment, the SHMT inhibitor has a structure of Formula (I): (I), or a pharmaceutically acceptable salt thereof, wherein, R1 is H or Cl, R2 is F or H, R3 is H or Cl. In one embodiment, Compound 2.2 as described herein has a structure of Formula (I) wherein R1 is H, R2 is F and R3 is H. In one embodiment, Compound 2.12 as described herein has a structure of Formula (I) wherein R1 is Cl, R2 is H and R is Cl. In one embodiment, Compound 2.2 and 2.12 is as described in Marani M., et al. Oncotarget. 2016 Jan. 26; 7 (4): 4570-83. In one embodiment, the SHMT inhibitor is AGF347. In one embodiment, the SHMT inhibitor has a structure of Formula II:

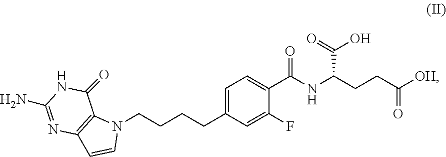 [SMILES: Nc1nc2ccn(CCCCc3ccc(C(=O)N[C@@H](CCC(=O)O)C(=O)O)c(F)c3)c2c(=O)[nH]1]

or a pharmaceutically acceptable salt thereof. In one embodiment, the SHMT inhibitor is SHIN1 (also herein SHIN-1 or RZ-2994). In another embodiment, the SHMT inhibitor has a structure of Formula III:

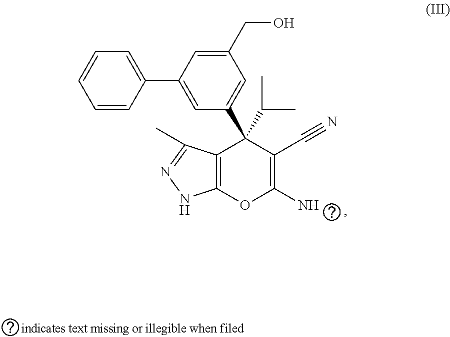 [SMILES: Cc1n[nH]c2c1[C@](c1cc(CO)cc(-c3ccccc3)c1)(C(C)C)C(C#N)=C(N)O2]

or a pharmaceutically acceptable salt thereof. In one embodiment, the SHMT inhibitor is SHIN2 (also herein SHIN-2 or (+) SHIN2). In one embodiment, the SHMT inhibitor has a structure of Formula IV:

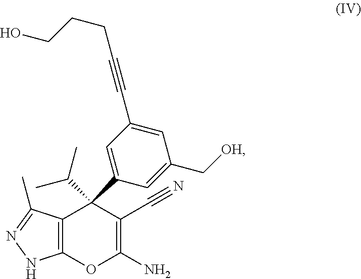 [SMILES: Cc1n[nH]c2c1[C@](c1cc(C#CCCCO)cc(CO)c1)(C(C)C)C(C#N)=C(N)O2]

or a pharmaceutically acceptable salt thereof. In one embodiment, the SHMT inhibitor is sertraline. In one embodiment, the SHMT inhibitor has a structure of Formula V:

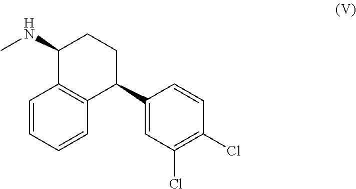 [SMILES: CN[C@H]1CC[C@@H](c2ccc(Cl)c(Cl)c2)c2ccccc21]

or a pharmaceutically acceptable salt thereof.

In one embodiment, the SMHT inhibitor is sulfite or S-sulfocysteine.

In one embodiment, one or more ferroptosis inducers or ferroptotic cell death inducers are used in the methods described herein. Ferroptosis inducers (FIN) used in the present disclosure can (i) inhibit system X_{c}⁻ and limit cysteine import; (ii) reduce or block GPX4 activity; (iii) degrade GPX4, bind to squalene synthase (SQS) and deplete antioxidant CoQ₁₀; and (iv) oxidize ferrous iron and lipidome directly and inactivate GPX4 directly. Non-limiting examples of ferroptosis inducers that inhibit system X_{c} and limit cysteine import include, erastin, sorafenib, sulfasalazine. Non-limiting examples of ferroptosis inhibitors include (1S,3R)-RSL3, ML210, ML162, JKE-1674, FIN56, DPI7, and DPI10. Non-limiting examples of ferroptosis inhibitors include ferrostatin-1; and liproxstatin-1.

In one embodiment, the ferroptosis inducer is a GPX4 inhibitor. GPX4 is a phospholipid hydroperoxidase that catalyzes the reduction of hydrogen peroxide and organic peroxides, and thereby protects cells against membrane lipid peroxidation, or oxidative stress. In one embodiment, a “GPX4 inhibitor” refers to any agent that reduces or blocks the activity of and/or reduces the abundance of the enzyme glutathione peroxidase 4 (GPX4).

A GPX4 inhibitor is either a direct or indirect inhibitor. In one embodiment, the GPX4 inhibitor inhibits GPX4 covalently. In one embodiment, the GPX4 inhibitor degrades or inactivate GPX4. In one embodiment, the GPX4 inhibitor is RSL3 (also herein RSL-3). In one embodiment, the GPX4 inhibitor has a structure of Formula VI:

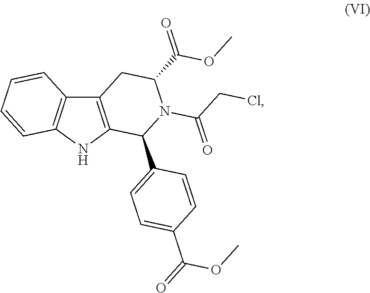 [SMILES: COC(=O)c1ccc([C@H]2c3[nH]c4ccccc4c3C[C@H](C(=O)OC)N2C(=O)CCl)cc1]

or a pharmaceutically acceptable salt thereof.

In one embodiment, the GPX4 inhibitor is erastin derivatives, ferroptosis-Inducer-56 (FIN56) and/or FINO₂. In one embodiment, the FIN56 has a structure of Formula VII:

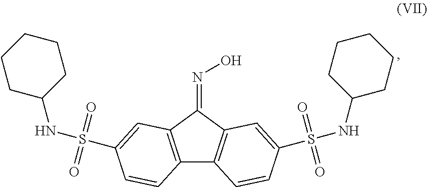 [SMILES: O=S(=O)(NC1CCCCC1)c1ccc2c(c1)C(=NO)c1cc(S(=O)(=O)NC3CCCCC3)ccc1-2]

or a pharmaceutically acceptable salt thereof. In one embodiment, FINO₂ has a structure of Formula VIII:

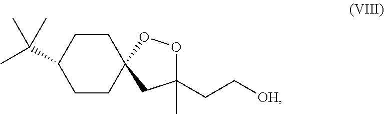

or a pharmaceutically acceptable salt thereof.

In one embodiment, the GPX4 inhibitor is ML-210. In one embodiment, the GPX inhibitor a structure of Formula IX:

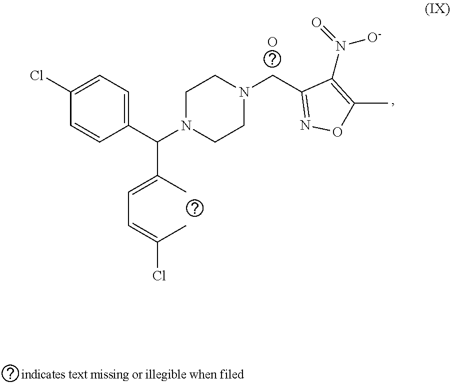

or a pharmaceutically acceptable salt thereof.

In one embodiment, the GPX4 inhibitor is ML-162. In one embodiment, the GPX4 inhibitor has a structure of Formula X:

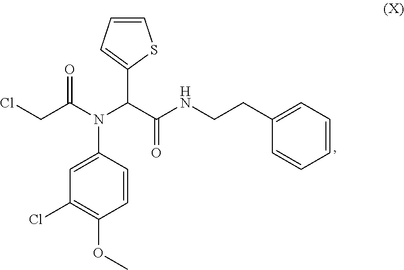 [SMILES: COc1ccc(N(C(=O)CCl)C(C(=O)NCCc2ccccc2)c2cccs2)cc1Cl]

or a pharmaceutically acceptable salt thereof. In one embodiment, the GPX4 inhibitor is altretamine. In one embodiment, the GPX4 inhibitor is a DPI7, DPI10, DPI12, DPI13, DPI17, DPI18, and/or DPI19. In one embodiment, the GPX4 inhibitor is JKE-1674.

### Methods of Use

Provided herein are methods of treating a cancer in a subject. Such methods include administering or providing to the subject one or more one-carbon metabolism inhibitors and one or more ferroptosis inducers. In one embodiment, at least one one-carbon metabolism inhibitor is a serine hydroxymethyltransferase (SHMT) inhibitor, a methylenetetrahydrofolate dehydrogenase (MTHFD) inhibitor, or a combination thereof.

In one embodiment, the present disclosure provides a method of treating cancer in a subject that includes administering to the subject: a) one or more one-carbon metabolism inhibitors; and b) one or more ferroptosis inducers, wherein the ferroptosis induced in the tumor is greater than the ferroptosis induced when the tumor is exposed to one or more ferroptosis inducers or one or more one-carbon metabolism inhibitors alone, and wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby enhancing ferroptosis in the tumor.

In another embodiment, the present disclosure provides a method of sensitizing cancer cells to ferroptosis that includes exposing the cancer cells to one or more one-carbon metabolism inhibitors, wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby sensitizing the cancer cells to ferroptosis.

In one aspect, ferroptosis is induced by administering one or more ferroptosis inducers. For example, in one aspect the ferroptosis inducer is a glutathione peroxidase 4 (GPX4) inhibitor. In one aspect, the SHMT inhibitor is an SHMT1 inhibitor, an SHMT2 inhibitor or an SHTM1/SHMT2 inhibitor. In one aspect, the MTHFD inhibitor is a MTHFD1 inhibitor, MTHFD2 inhibitor, an MTHFD1L inhibitor or a combination thereof.

Also provided herein are methods of enhancing ferroptosis in a tumor. Such methods include exposing the tumor to one or more one-carbon metabolism inhibitors and one or more ferroptosis inducers. In one embodiment, the ferroptosis induced in the tumor is greater than the ferroptosis induced when the tumor is exposed to one or more ferroptosis inducers inhibitors or to one or more one-carbon metabolism inhibitors alone. In one embodiment, the combination of one or more one-carbon metabolism inhibitors and one or more ferroptosis inducers induces ferroptosis by about 0-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90% or more when compared to one or more ferroptosis inducers inhibitors or one or more one-carbon metabolism inhibitors alone.

The present disclosure also provides for a method of sensitizing cancer cells to ferroptosis including exposing cancer cells to one or more one-carbon metabolism inhibitors. The method includes sensitizing the cancer cells to ferroptosis by about 0-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90% or more when compared to one or more ferroptosis inducers inhibitors or one or more one-carbon metabolism inhibitors alone.

In one embodiment, the one-carbon metabolism inhibitors and one or more ferroptosis inducers are administered simultaneously to the subject. In one embodiment, one-carbon metabolism inhibitors and one or more ferroptosis inducers are administered sequentially to the subject. In one embodiment, the one-carbon metabolism inhibitors are administered first. In one embodiment, the one or more ferroptosis inducers are administered first. In one embodiment, the two inhibitors in the sequence are administered within minutes, hours, days, or weeks of each other. The methods of the present disclosure are used to treat a cancer such as leukemia, breast cancer, kidney cancer, brain cancer, non-small cell lung cancer, prostate cancer, colon cancer, cervical cancer, or fibrosarcoma.

As used herein, the term “treatment” refers to an approach or regimen designed to improve or alleviate symptoms of a disease, sickness, or infirmity. Treatment can lead to reduction in pain, improvement of quality of life, or decrease in size, number, or distribution of a tumor, cancer, or cancerous cells. Treating cancer can also occur when symptoms or tests for cancer or cancerous cells are nonexistent or improved, or when symptoms or tests for cancer or cancerous cells do not worsen, or stabilize. The term “effective amount” refers to the amount of a compound, composition, or formulation that is sufficient to treat a condition or disease, to produce desirable effects or results, or to reduce, ease, or arrest symptoms of a condition or disease. “Effective amount” is used interchangeably with the term “therapeutically effective amount”.

In one aspect, the cancer is a tumor. In one aspect, the tumor is a carcinoma. In one embodiment, the tumor is a solid tumor. A solid tumor includes sarcoma, melanoma, carcinoma, or other solid tumor cancer. Sarcomas include, but are not limited to, chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, choriocarcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectatic sarcoma.

Melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungual melanoma, or superficial spreading melanoma. Further carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidemoid carcinoma, carcinoma mucosum, mucous carcinoma, carcinoma myxomatosis, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatoides, Schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectoides, transitional cell carcinoma, carcinoma tuberosum, tuberous carcinoma, verrucous carcinoma, or carcinoma viflosum.

Additional cancers that are treated according to the methods disclosed herein include, e.g., leukemia, Hodgkin's disease, non-Hodgkin's lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In one aspect, the cancer is relapsed, refractory, or refractory following at least one prior therapy including administration of at least one anti-cancer agent.

In one aspect, the cancer is selected from fibrosarcoma, lung squamous cell carcinoma, lung adenocarcinoma, renal cell carcinoma, breast adenocarcinoma, colorectal adenocarcinoma, endocervical adenocarcinoma, or T acute lymphoblastic leukemia.

In one embodiment, the present disclosure provides a method of treating cancer in a subject that includes administering to the subject: a) one or more one-carbon metabolism inhibitors; and b) one or more ferroptosis inducers, thereby treating cancer in the subject. In one embodiment, the one-carbon metabolism inhibitor is a serine hydroxymethyltransferase (SHMT) inhibitor, a methylenetetrahydrofolate dehydrogenase (MTHFD) inhibitor or a combination thereof.

In one embodiment, the present disclosure provides a method of enhancing ferroptosis in a tumor that includes exposing the tumor to one or more one-carbon metabolism inhibitors and one or more ferroptosis inducers. In one aspect, the ferroptosis induced in the tumor is greater than the ferroptosis induced when the tumor is exposed to one or more ferroptosis inducers inhibitors or one or more one-carbon metabolism inhibitors alone. In one aspect, the one-carbon metabolism inhibitor is a serine hydroxymethyltransferase (SHMT) inhibitor, a methylenetetrahydrofolate dehydrogenase (MTHFD) inhibitor or a combination thereof.

In another embodiment, the present disclosure provides a method of sensitizing cancer cells to ferroptosis that includes exposing the cancer cells to one or more one-carbon metabolism inhibitors, thereby sensitizing the cancer cells to ferroptosis. In one aspect, the one-carbon metabolism inhibitor is a serine hydroxymethyltransferase (SHMT) inhibitor, a methylenetetrahydrofolate dehydrogenase (MTHFD) inhibitor or a combination thereof.

In one aspect, ferroptosis is induced by administering one or more ferroptosis inducers. For example, in one aspect the ferroptosis inducer is a glutathione peroxidase 4 (GPX4) inhibitor. In one aspect, the SHMT inhibitor is an SHMT1 inhibitor, an SHMT2 inhibitor or an SHTM1/SHMT2 inhibitor. In one aspect, the MTHFD inhibitor is a MTHFD1 inhibitor, MTHFD2 inhibitor, an MTHFD1L inhibitor or a combination thereof.

In one aspect, the one or more one-carbon metabolism inhibitors and the one or more ferroptosis inducers are administered at the same time.

In one aspect, the one or more one-carbon metabolism inhibitors and the one or more ferroptosis inducers are administered sequentially.

In one aspect, the one or more SHMT inhibitor is SHIN2, AGF347, SHIN1, sertraline, Compound 2.12, Compound 2.2, sulfite, S-sulfocysteine, or a combination thereof.

In one aspect, the one or more GPX4 inhibitors is RSL3, FIN56, FINO₂, ML-210, M-162, JKE-1674 or a combination thereof.

In one aspect, administering the one or more one-carbon metabolism inhibitors and the one or more ferroptosis inducers results in an increase in ferroptosis.

In one aspect, the cancer is leukemia, breast cancer, kidney cancer, brain cancer, non-small cell lung cancer, prostate cancer, colon cancer, cervical cancer, or fibrosarcoma.

In one embodiment, the present disclosure provides a pharmaceutical composition that includes a) one or more one-carbon metabolism inhibitors; and b) one or more ferroptosis inducers. In one aspect, the one-carbon metabolism inhibitor is a serine hydroxymethyltransferase (SHMT) inhibitor, a methylenetetrahydrofolate dehydrogenase (MTHFD) inhibitor or a combination thereof. In one aspect, the SHMT inhibitor is an SHMT1 inhibitor, an SHMT2 inhibitor or an SHTM1/SHMT2 inhibitor. In one aspect, the SHMT inhibitor is SHIN2, AGF347, SHIN1, sertraline, Compound 2.12, Compound 2.2, sulfite, S-sulfocysteine, or a combination thereof. In one aspect, the MTHFD inhibitor is a MTHFD1 inhibitor, MTHFD2 inhibitor, an MTHFD1L inhibitor or a combination thereof. In one aspect, the ferroptosis inducer is a glutathione peroxidase 4 (GPX4) inhibitor. In one aspect, the GPX4 inhibitor is RSL3, FIN56, ML-210, M-162, JKE-1674 or a combination thereof. In another aspect, a unit dose of the SHMT inhibitor when present is about 0.01 mg to about 1 g, a unit dose of the MTHFD inhibitor when present is about 0.01 mg to about 1 g, and a unit dose of the GPX4 inhibitor when present is about 0.01 mg to about 1 g.

In one aspect, the methods disclosed herein are combined with standard of care for cancer therapy. In one aspect, standard of care includes, but is not limited to, chemotherapy, radiotherapy, administering immunotherapy, administering targeted therapy, and combination thereof.

In one aspect, the methods disclosed herein reduce the cancer burden. In one aspect, the cancer burden is reduced by at least about 10%, at least about 20%, at least about 30%, at least about 40%, or about 50% as compared to the cancer burden prior to the administration of the one-carbon metabolism inhibitor e.g., SHMT inhibitor and/or the ferroptosis inducer.

In one aspect, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration of the one-carbon metabolism inhibitor e.g., SHMT inhibitor and/or the ferroptosis inducer.

In one aspect, the subject exhibits stable disease about one month, about 2 months, about 3 months, about 4 months, about 5 months, about 6 months, about 7 months, about 8 months, about 9 months, about 10 months, about 11 months, about one year, about eighteen months, about two years, about three years, about four years, or about five years after the initial administration of the one-carbon metabolism inhibitor e.g., SHMT inhibitor and/or the ferroptosis inducer. The term “stable disease” refers to a diagnosis for the presence of a cancer, however the cancer has been treated and remains in a stable condition, i.e., one that that is not progressive, as determined, e.g., by imaging data and/or best clinical judgment. The term “progressive disease” refers to a diagnosis for the presence of a highly active state of a cancer, i.e., one that has not been treated and is not stable or has been treated and has not responded to therapy, or has been treated and active disease remains, as determined by imaging data and/or best clinical judgment.

Pharmaceutical compositions within the scope of the present disclosure include all compositions where a one-carbon metabolism inhibitor (e.g., SHMT inhibitor) and/or a ferroptosis inducer (e.g., a GPX4 inhibitor) are combined with one or more pharmaceutically acceptable carriers. In one aspect, the SHMT inhibitor and/or the GPX4 inhibitor are present in the composition in an amount that is effective to achieve an intended therapeutic purpose. Typically, the SHMT inhibitor and/or the GPX4 inhibitor, individually or combined, are administered to a mammal, e.g., a human, at a dose of from about 0.0025 to about 1500 mg per kg body weight of the mammal, or an equivalent amount of a pharmaceutically acceptable salt or solvate thereof, per day, to treat the particular disorder. A useful dose of the SHMT inhibitor and/or the GPX4 inhibitor, individually or combined, administered to a mammal, is from about 0.0025 to about 50 mg per kg body weight of the mammal, or an equivalent amount of the pharmaceutically acceptable salt or solvate thereof.

In one embodiment, the dose of the SHMT inhibitor is about 0.0025 to 0.1 mg/kg, about 0.1 to 20 mg/kg, about 0.1 to 1 mg/kg, about 1 to 10 mg/kg, about 1 to 5 mg/kg, about 10 to 20 mg/kg, about 20 to 30 mg/kg, about 30 to 40 mg/kg, about 40 to 50 mg/kg, about 50 to 60 mg/kg, about 60 to 70 mg/kg, about 70 to 80 mg/kg, about 80 to 90 mg/kg, about 90 to 100 mg/kg, about 0.0025 to about 1500 mg/kg, and all ranges and values in between the suggested ranges. In one embodiment, the dose of the SHMT inhibitor is about 0.0025 mg/kg, about 0.01 mg/kg, about 0.05 mg/kg, about 0.1 mg/kg, about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg. The dose is administered every day, every other day, or every week.

In one embodiment, the dose of the MTHFD inhibitor is about 0.0025 to 0.1 mg/kg, about 0.1 to 20 mg/kg, about 0.1 to 1 mg/kg, about 1 to 10 mg/kg, about 1 to 5 mg/kg, about 10 to 20 mg/kg, about 20 to 30 mg/kg, about 30 to 40 mg/kg, about 40 to 50 mg/kg, about 50 to 60 mg/kg, about 60 to 70 mg/kg, about 70 to 80 mg/kg, about 80 to 90 mg/kg, about 90 to 100 mg/kg, about 0.0025 to about 1500 mg/kg, and all ranges and values in between the suggested ranges. In one embodiment, the dose of the SHMT inhibitor is about 0.0025 mg/kg, about 0.01 mg/kg, about 0.05 mg/kg, about 0.1 mg/kg, about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg. The dose is administered every day, every other day, or every week.

In one embodiment, the dose of the GPX4 inhibitor is about 0.0025 to 0.1 mg/kg, about 0.1 to 20 mg/kg, about 0.1 to 1 mg/kg, about 1 to 10 mg/kg, about 1 to 5 mg/kg, about 10 to 20 mg/kg, about 20 to 30 mg/kg, about 30 to 40 mg/kg, about 40 to 50 mg/kg, about 50 to 60 mg/kg, about 60 to 70 mg/kg, about 70 to 80 mg/kg, about 80 to 90 mg/kg, about 90 to 100 mg/kg, about 0.0025 to about 1500 mg/kg, and all ranges and values in between the suggested ranges. In one embodiment, the dose of the SHMT inhibitor is about 0.0025 mg/kg, about 0.01 mg/kg, about 0.05 mg/kg, about 0.1 mg/kg, about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg. The dose is administered every day, every other day, or every week.

A unit dose includes from about 0.01 mg to about 1 g of the SHMT inhibitor and/or the GPX4 inhibitor, individually or combined; e.g., about 0.01 mg to about 500 mg, about 0.01 mg to about 250 mg, about 0.01 mg to about 100 mg, 0.01 mg to about 50 mg, e.g., about 0.1 mg to about 10 mg of the SHMT inhibitor and/or the GPX4 inhibitor. In one aspect, the unit does includes about 0.01 mg, about 0.05 mg, about 0.1 mg, about 0.5 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, or about 510 mg of the SHMT inhibitor.

In one aspect, the unit does includes about 0.01 mg, about 0.05 mg, about 0.1 mg, about 0.5 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, or about 510 mg of the GPX4 inhibitor.

In one aspect, the unit does includes about 0.01 mg, about 0.05 mg, about 0.1 mg, about 0.5 mg, about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, or about 510 mg of the MTHFD inhibitor.

The unit dose is administered one or more times daily, e.g., as one or more tablets or capsules, each containing from about 0.01 mg to about 1 g of the SHMT inhibitor and/or the GPX4 inhibitor, or an equivalent amount of a pharmaceutically acceptable salt or solvate thereof.

A pharmaceutical composition including the SHMT inhibitor and/or the GPX4 inhibitor is administered to any subject, e.g., a cancer patient in need thereof, that experiences the beneficial effects of the SHMT inhibitor and/or the GPX4 inhibitor. Such subjects include mammals, e.g., humans and companion animals, although the disclosure is not intended to be so limited. In one aspect, the subject is a human.

In one aspect, the SHMT inhibitor is administered at the same time as the GPX4 inhibitor. In one aspect, the SHMT inhibitor is administered at a different time than the GPX4 inhibitor. In another aspect, the SHMT inhibitor and the GPX4 inhibitor are administered sequentially. In an aspect, the SHMT inhibitor is administered followed by administration of the GPX4 inhibitor. In another aspect, the GPX4 inhibitor is administered followed by administration of the SHMT2 inhibitor.

A pharmaceutical composition of the present disclosure is administered by any means that achieves its intended purpose. For example, administration is by the oral, parenteral, subcutaneous, intravenous, intramuscular, intraperitoneal, transdermal, intranasal, transmucosal, rectal, intravaginal, or buccal route, or by inhalation. The dosage administered and route of administration vary, depending upon the circumstances of the particular subject, and considering such factors as age, gender, health, and weight of the recipient, the condition or disorder to be treated, the nature of any concurrent treatments, the frequency of treatment, and the nature of the therapeutic effect desired.

In one aspect, a pharmaceutical composition of the present disclosure is administered orally. In one aspect, a pharmaceutical composition of the present disclosure is administered orally and is formulated into tablets, dragees, capsules, or an oral liquid preparation. In one aspect, the oral formulation includes extruded multiparticulates including the SHMT inhibitor and/or the GPX4 inhibitor.

Alternatively, a pharmaceutical composition of the present disclosure is administered rectally and is formulated in a suppository.

Alternatively, a pharmaceutical composition of the present disclosure is administered by injection.

Alternatively, a pharmaceutical composition of the present disclosure is administered transdermally.

Alternatively, a pharmaceutical composition of the present disclosure is administered by inhalation or by intranasal or transmucosal administration.

Alternatively, a pharmaceutical composition of the present disclosure is administered by the intravaginal route.

A pharmaceutical composition of the present disclosure contains from about 0.01 to 99 percent by weight, e.g., from about 0.25 to 75 percent by weight, of the SHMT inhibitor, MTHFD inhibitor, and/or the GPX4 inhibitor, individually or combined, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the SHMT inhibitor and/or the GPX4 inhibitor, individually or combined.

In one aspect, the pharmaceutical composition contains from about 0.01 to 0.1, about 0.1 to 0.5, about 0.5 to 1, about 1 to 5, about 5 to 10, about 10 to 15, about 15 to 20, about 20 to 25, about 25 to 30, about 30 to 40, about 40-50, about 50 to 60, about 60 to 70, about 70 to 80, about 80 to 90, or about 90 to 99 percent by weight of the SHMT inhibitor. In another aspect, the pharmaceutical composition contains about 1%, about 5%, about 10%, about 15%, about 20%, about 25%, about 30%, about 35%, about 40%, about 45%, about 50%, about 55%, about 60%, about 65%, about 70%, or about 75% by weight of the SHMT inhibitor.

In one aspect, the pharmaceutical composition contains from about 0.01 to 0.1, about 0.1 to 0.5, about 0.5 to 1, about 1 to 5, about 5 to 10, about 10 to 15, about 15 to 20, about 20 to 25, about 25 to 30, about 30 to 40, about 40-50, about 50 to 60, about 60 to 70, about 70 to 80, about 80 to 90, or about 90 to 99 percent by weight of the MTHFD inhibitor. In some aspects, the pharmaceutical composition contains about 1%, about 5%, about 10%, about 15%, about 20%, about 25%, about 30%, about 35%, about 40%, about 45%, about 50%, about 55%, about 60%, about 65%, about 70%, or about 75% by weight of the MTHFD inhibitor.

In one aspect, the pharmaceutical composition contains from about 0.01 to 0.1, about 0.1 to 0.5, about 0.5 to 1, about 1 to 5, about 5 to 10, about 10 to 15, about 15 to 20, about 20 to 25, about 25 to 30, about 30 to 40, about 40-50, about 50 to 60, about 60 to 70, about 70 to 80, about 80 to 90, or about 90 to 99 percent by weight of the GPX4 inhibitor. In some aspects, the pharmaceutical composition contains about 1%, about 5%, about 10%, about 15%, about 20%, about 25%, about 30%, about 35%, about 40%, about 45%, about 50%, about 55%, about 60%, about 65%, about 70%, or about 75% by weight of the GPX4 inhibitor.

Pharmaceutical compositions for oral use are obtained by combining at least one of the SHMT inhibitor, the MTHFD inhibitor, and the GPX4 inhibitor with solid excipients, and optionally grinding the resulting mixture and processing the mixture of granules after adding suitable excipients, if desired or necessary, to obtain tablets or dragee cores.

In one aspect, an effective dosage of at least one of the SHMT inhibitor, the MTHFD inhibitor, and the GPX4 inhibitor is administered to a subject in need thereof. In another aspect, the effective dosage of the SHMT inhibitor is about 0.0025 to about 1500 mg/kg, the effective dosage of the MTHFD inhibitor is about 0.0025 to about 1500 mg/kg, and the effective dosage of the GPX4 inhibitor is about 0.0025 to about 1500 mg/kg.

Suitable excipients include fillers such as saccharides (for example, lactose, sucrose, mannitol, or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents are added, such as the above-mentioned starches and also carboxymethyl-starch, cross-linked polyvinyl pyrrolidone, agar, or alginic acid or a salt thereof, such as sodium alginate.

Auxiliaries are typically flow-regulating agents and lubricants such as, for example, silica, talc, stearic acid, or salts thereof (e.g., magnesium stearate or calcium stearate), and polyethylene glycol. Dragee cores are provided with suitable coatings that are resistant to gastric juices. For this purpose, concentrated saccharide solutions are used, which optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate is used. Dye stuffs or pigments are added to the tablets or dragee coatings, for example, for identification or in order to characterize combinations of active compound doses.

Examples of other pharmaceutical preparations that are used orally include push-fit capsules made of gelatin, or soft, sealed capsules made of gelatin and a plasticizer such as glycerol or sorbitol. The push-fit capsules contain SHMT inhibitor and/or the GPX4 inhibitor in the form of granules, which are mixed with fillers such as lactose, binders such as starches, and/or lubricants such as talc or magnesium stearate and, optionally, stabilizers, or in the form of extruded multiparticulates. In soft capsules, the SHMT inhibitor and/or the GPX4 inhibitor are preferably dissolved or suspended in suitable liquids, such as fatty oils or liquid paraffin. In addition, stabilizers are added.

Possible pharmaceutical preparations for rectal administration include, for example, suppositories, which include SHMT inhibitor and/or the GPX4 inhibitor with a suppository base. Suitable suppository bases include natural and synthetic triglycerides, and paraffin hydrocarbons, among others. It is also possible to use gelatin rectal capsules containing SHMT inhibitor and/or the GPX4 inhibitor with a base material such as, for example, a liquid triglyceride, polyethylene glycol, or paraffin hydrocarbon.

Suitable formulations for parenteral administration include aqueous solutions of the SHMT inhibitor and/or the GPX4 inhibitor in a water-soluble form such as, for example, a water-soluble salt, alkaline solution, or acidic solution. Alternatively, a suspension of SHMT inhibitor and/or the GPX4 inhibitor is prepared as an oily suspension, a water-in-oil emulsion, or an oil-in-water emulsion. Suitable lipophilic solvents or vehicles for such as suspension and emulsions include fatty oils (for example, sesame oil), synthetic fatty acid esters (for example, ethyl oleate), triglycerides, or a polyethylene glycol such as polyethylene glycol-400 (PEG-400). An aqueous suspension contains one or more substances to increase the viscosity of the suspension, including, for example, sodium carboxymethyl cellulose, sorbitol, and/or dextran. The suspension optionally contains stabilizers.

The present disclosure provides kits which include one-carbon metabolism inhibitors and/or ferroptosis inducers. In one aspect, the present disclosure provides kits which include the SHMT inhibitor and/or the GPX4 inhibitor (or a pharmaceutical composition thereof) packaged in a manner that facilitates their use to practice methods of the present disclosure. In one aspect, the kit includes the SHMT inhibitor and/or the GPX4 inhibitor (or a pharmaceutical composition thereof) packaged in a container, such as a sealed bottle or vessel, with a label affixed to the container or included in the kit that describes use of the SHMT inhibitor and/or the GPX4 inhibitor or the pharmaceutical composition thereof to practice the method of the disclosure. In one aspect, SHMT inhibitor and/or the GPX4 inhibitor or the pharmaceutical composition thereof is packaged in a unit dosage form. The kit further includes a device suitable for administering the composition according to the intended route of administration.
Dixon, S. J., et al., Cell 149, 1060-1072 (2012)
Stockwell, B. R., et al., Cell 171, 273-285 (2017)
Jiang, L., et al., Nature 520, 57-62 (2015)
Zhang, Y., et al., Nat Cell Biol 20, 1181-1192 (2018)
Yang, W. S., et al., Cell 156, 317-331 (2014)
Doll, S., et al., Nature 575, 693-698 (2019)
Koppula, P., Zhuang, L. & Gan, B., Protein Cell (2020)

### EXAMPLES

The following examples are provided to further illustrate the embodiments of the present invention but are not intended to limit the scope of the invention. While they are typical of those that might be used, other procedures, methodologies, or techniques known to those skilled in the art may alternatively be used.

### Example 1: Sulfite Treatment Sensitizes Cancer Cells to GPX4 Inhibition Induced Ferroptosis

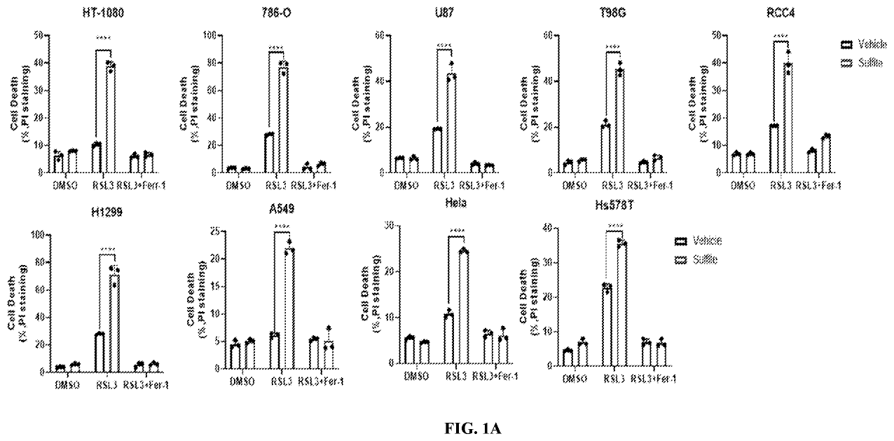

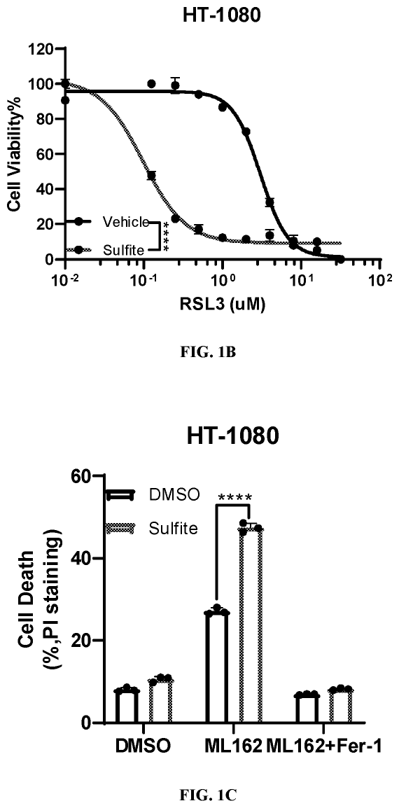

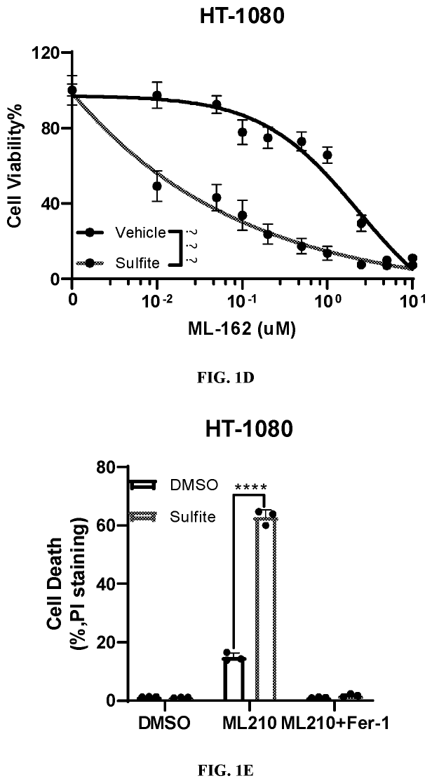

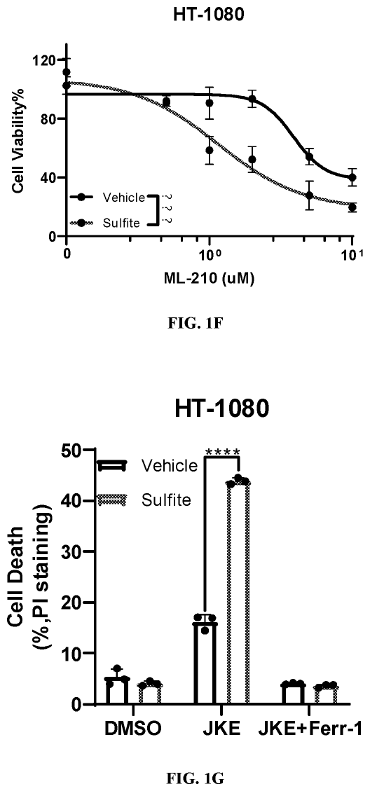

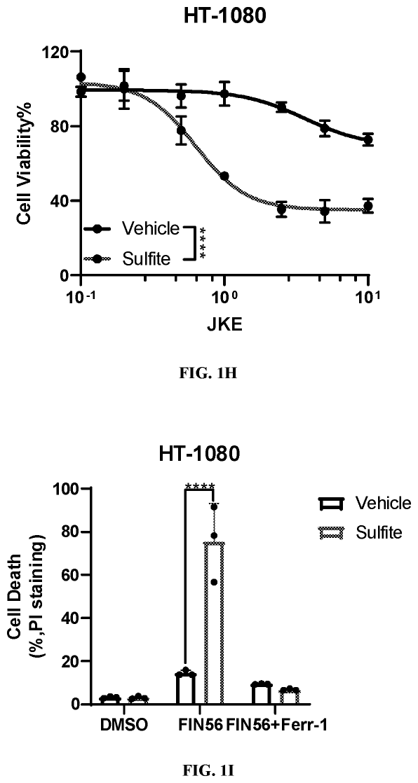

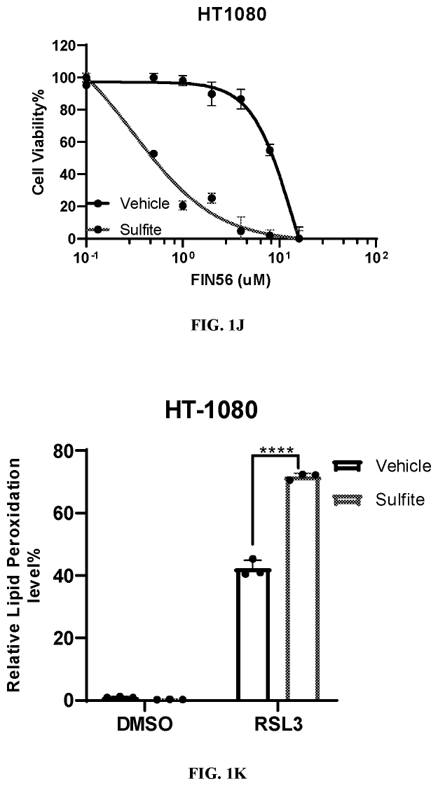

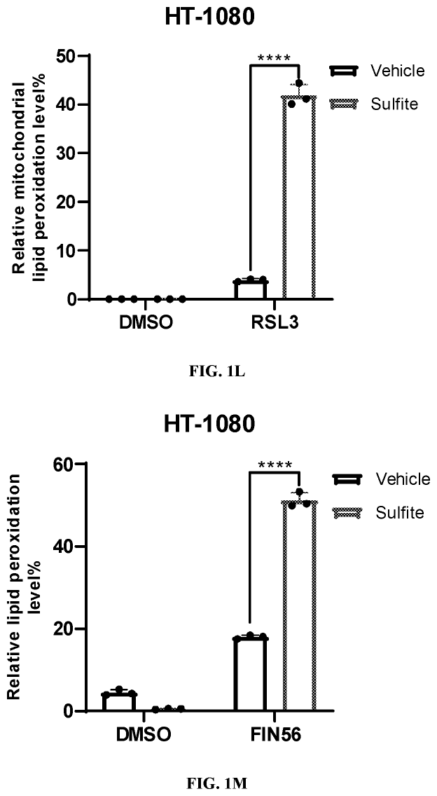

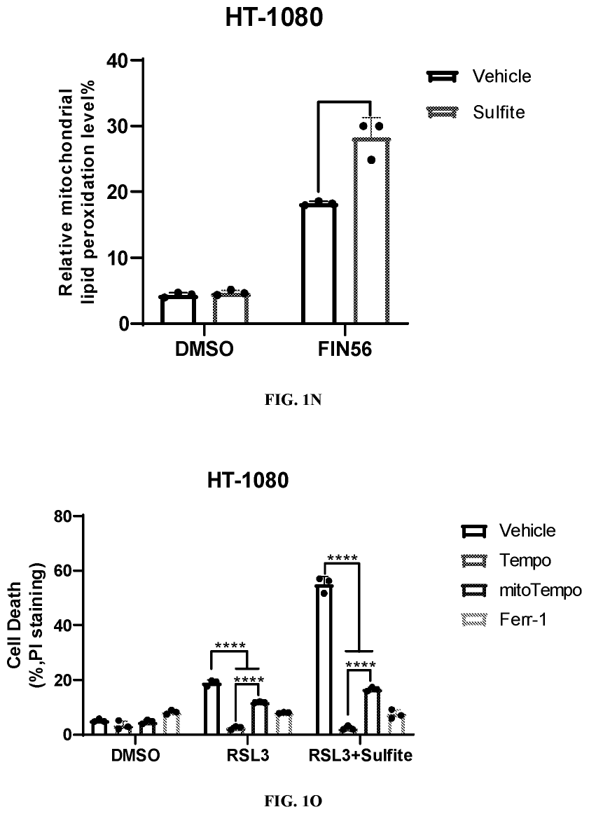

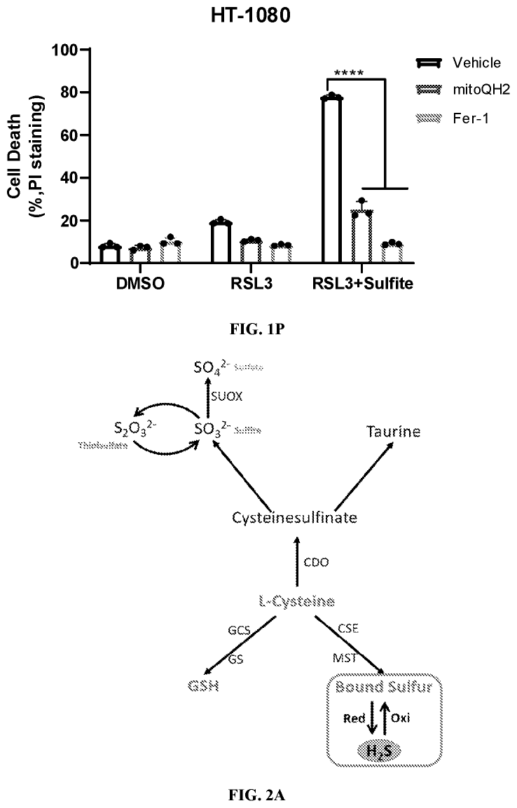

Initially, the effect of sulfite on ferroptosis was studied. HT-1080, 786-O, U87, T98G, RCC4, H1299, A549, Hela and Hs578T were treated with DMSO control, ferroptosis inducer RSL3 or a combination of RSL3 and Fer-1 is a ferroptosis inhibitor. Each group was treated with vehicle alone or with sulfite. For HT-1080 cells, the concentration of inhibitors used was as follows: RSL3 0.5 μm, Fer-1 5 μm, Sulfite 0.5 mM, ML-162 0.2 μm, JKE 2 μm, FIN56 5 μm. For 786-O cells, the concentration of inhibitors used was as follows: RSL3 0.2 μm, Sulfite 0.5 mM. For U87 cells, the concentration of inhibitors used was as follows: RSL3 0.5 μm Sulfite 0.5 mM. For T98G cells, the concentration of inhibitors used was as follows: RSL3 2 μm Sulfite 1.0 mM. For RCC4 cells, the concentration of inhibitors used was as follows: RSL3 0.2 μm, Sulfite 0.5 mM. For H1299 cells, the concentration of inhibitors used was as follows: RSL3 0.5 μm, Sulfite 0.5 mM. For A549 cells, the concentration of inhibitors used was as follows: RSL3 2.0 μm, Sulfite 2 mM. For Hela cells, the concentration of inhibitors used was as follows: RSL3 1.0 μm, Sulfite 1 mM. For Hs578T cells, the concentration of inhibitors used was as follows: RSL3 2.0 μm, Sulfite 2 mM. Significantly, treatment with sulfite alone did not show induction of ferroptosis but the combination of RSL3 and sulfite induced statistically significant ferroptosis (FIG. 1A). The ferroptosis induced by the combination of RSL3 and sulfite could be rescued by Fer-1 showing that the effect is specific to ferroptosis.

In HT-1080 cells, sulfite reduced cell viability when combined with different concentrations of RSL3 (FIG. 1B). The effect of different ferroptosis inducers ML-162 (FIG. 1C, FIG. 1D), ML-210 (FIG. 1E, FIG. 1F), JKE (FIG. 1G, FIG. 1H) and FIN56 (FIG. 1I and FIG. 1J) was similarly enhanced by sulfite treatment in HT-1080 cells.

Consistent with a ferroptosis mechanism of action, relative lipid peroxidation levels (FIG. 1K) and relative mitochondrial lipid peroxidation level (FIG. 1L) were significantly increased with RSL3, and sulfite treatment compared to treatment with RSL3 alone. Similar results were seen when FIN56 was used as the ferroptosis inducer (FIG. 1M and FIG. 1N).

Cell death induced by RSL3 plus sulfite could be rescued by mitochondrial specific lipid peroxidation neutralizer, but not RSL3 treated alone (FIG. 1O and FIG. 1P).

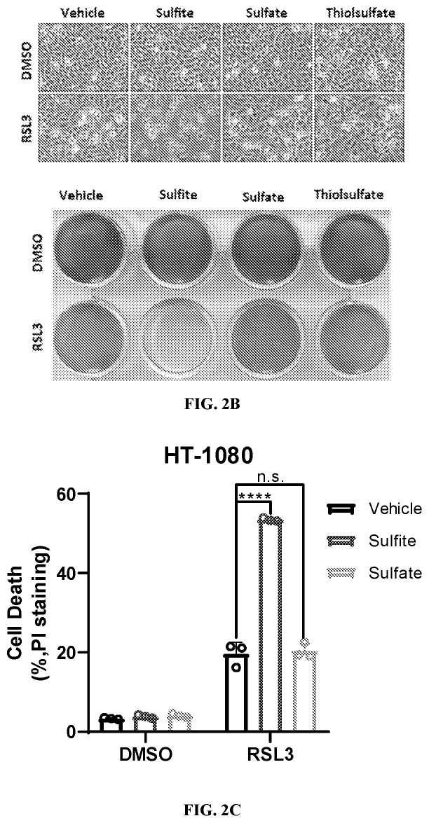

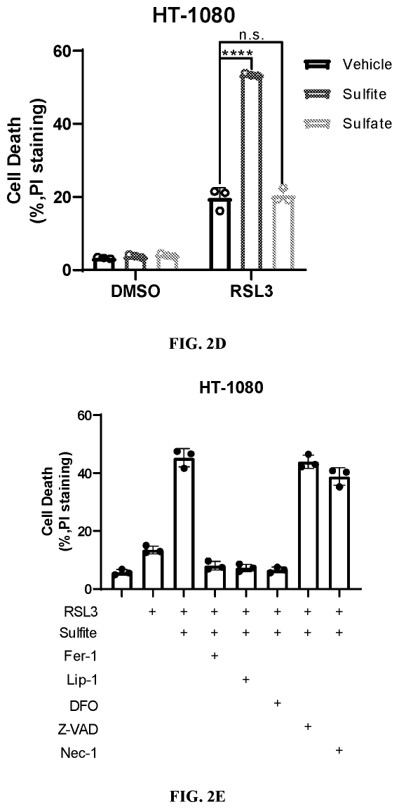

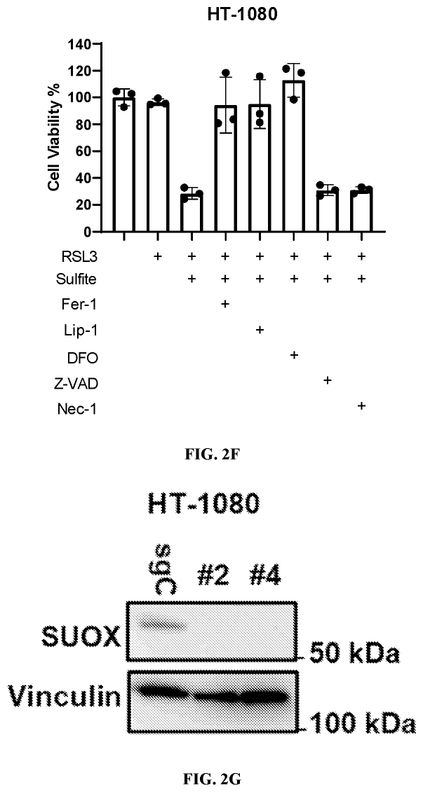

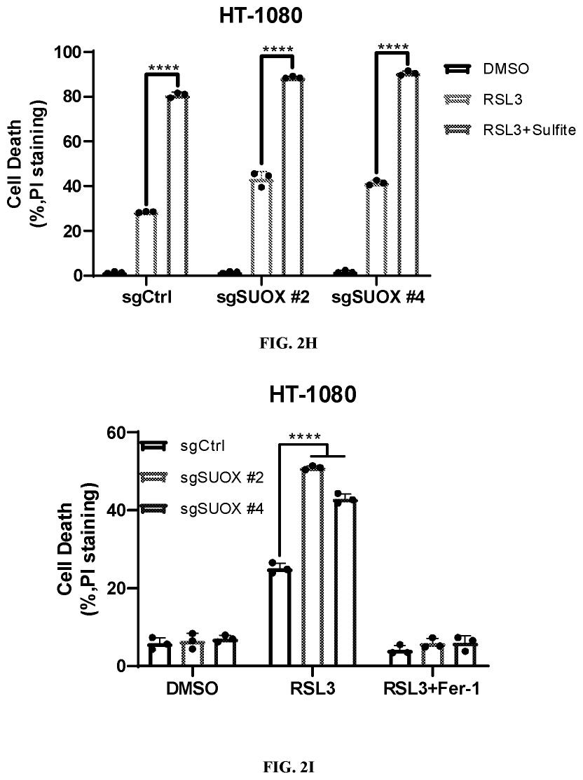

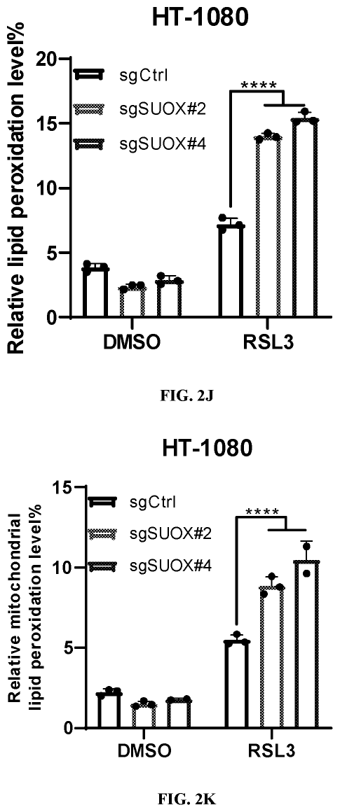

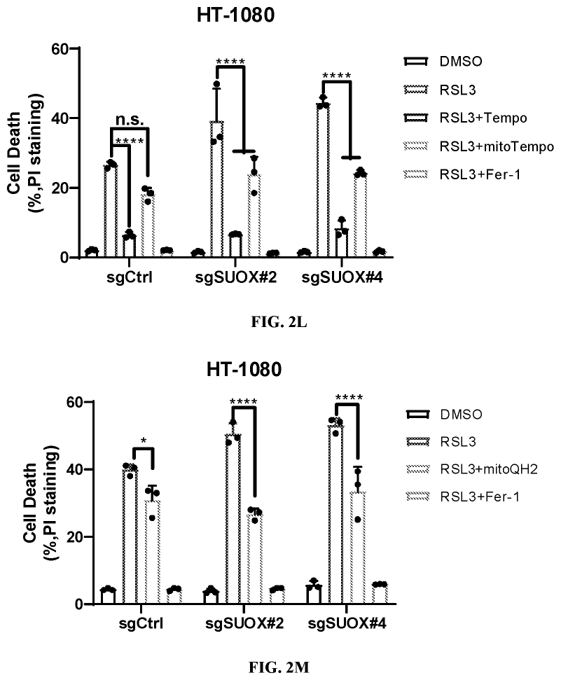

The pathway involving sulfite is shown in FIG. 2A. Sulfite is reversibly converted to thiosulfate. Sulfite is converted to sulfate. The effect of sulfite, sulfate and thiosulfate was tested in cancer cells in the presence or absence of RSL3. Cells were treated with sulfite, sulfate, and thiosulfate at a concentration of 0.5 mM. Sulfite and to a very small extent, thiosulfate resulted in cell death as observed visually (FIG. 2B). Accordingly, cell death observed with thiosulfate was significantly more than the cell death observed with vehicle control upon treatment with RSL3, but less than the cell death observed with sulfite (FIG. 2C). Sulfate did not synergistically increase RSL3 induced cell death (FIG. 2D).

As show in FIG. 2E and FIG. 2F, RSL3 plus sulfite induced cell death or decrease in cell viability which was be rescued by ferroptosis inhibitor ferrostatin-1, liprostatin-1 and deferoxamine but not apoptosis nor necrosis inhibitor.

Sulfite oxidase (SUOX) is an enzyme in the mitochondria that sulfite to sulfate. To test the role of sulfite oxidase in ferroptosis, SUOX was knocked out using CRISPR-Cas9-mediated gene knockout (FIG. 2G). RSL3 and sulfite treatment induced significantly more cell death than just RSL3 treatment alone in SUOX knockout cells HT-1080 cells (FIG. 2H). Greater cell death was observed in SUOX knockout HT-1080 cells treated with RSL3 than sg control cells. The impact of SUOX knockout on cell death was rescued upon treatment adding Fer-1 and RSL3 showing that the effect is ferroptosis specific (FIG. 2I). This effect was also mirrored in the relative lipid peroxidation level % (FIG. 2J) and relative mitochondrial lipid peroxidation level % (FIG. 2K).

As shown in FIG. 2L and FIG. 2M, cell death induced by RSL3 in SUOX knock out cells could be rescued by mitochondrial specific lipid peroxidation neutralizer, but not in wild type cells.

### Example 2: Sulfite Treatment Disturbs NADP+/NADPH Balance by Inhibiting One-Carbon Metabolism

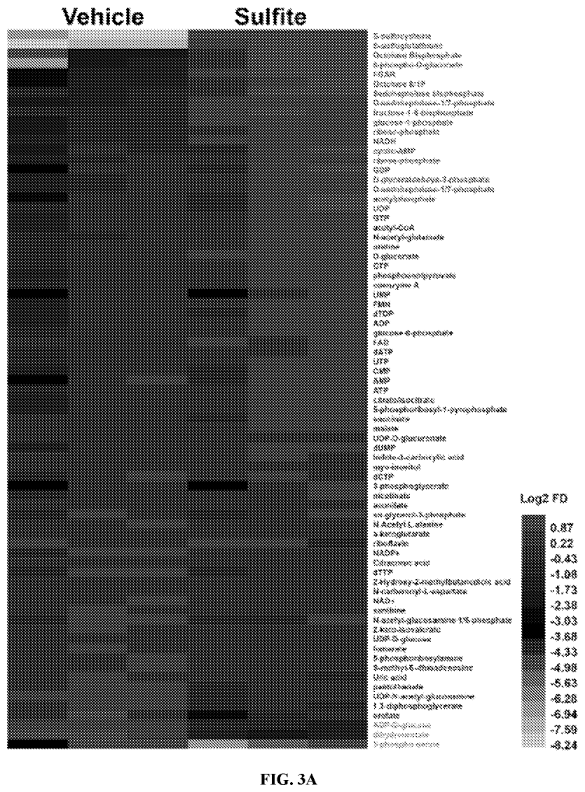

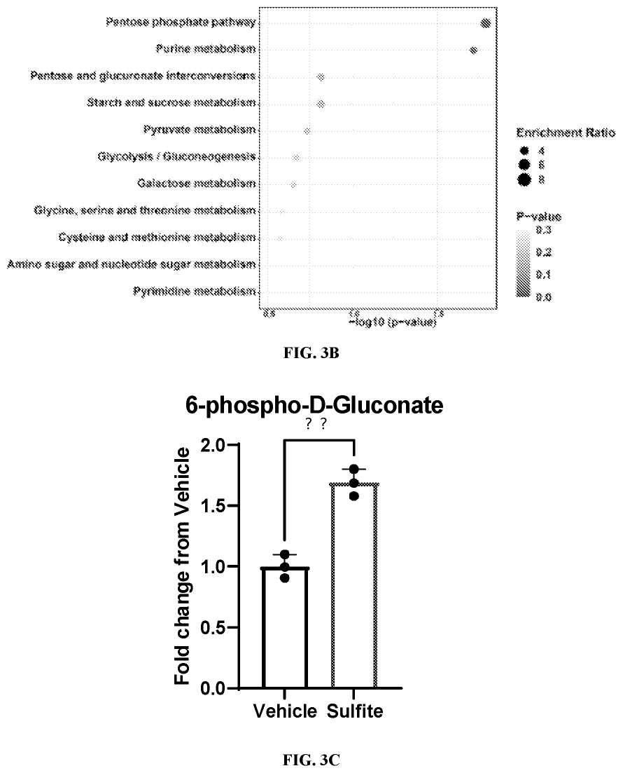

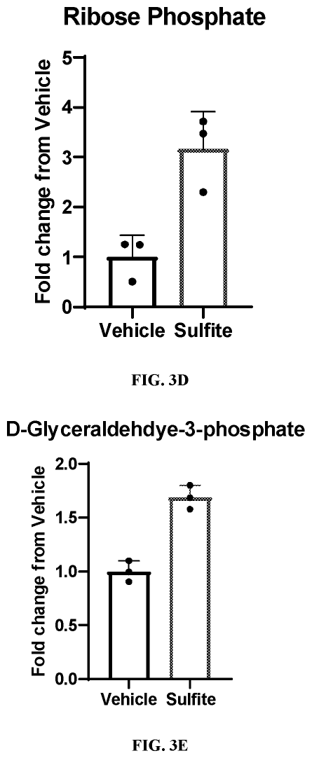

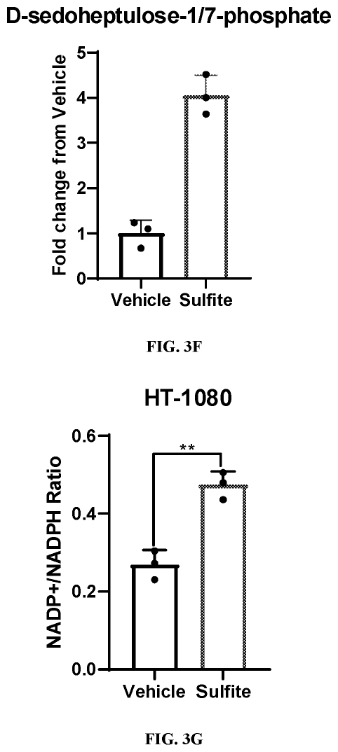

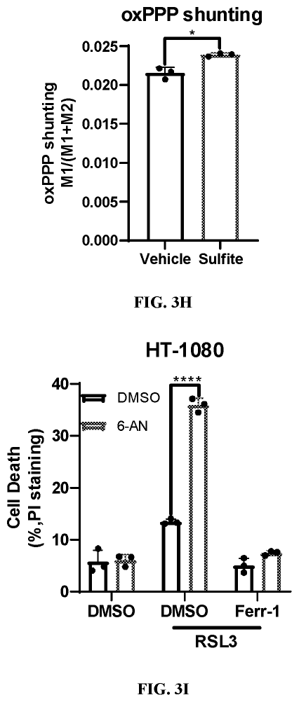

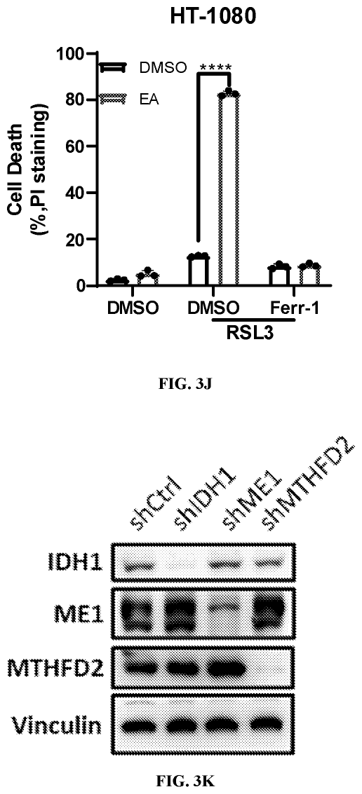

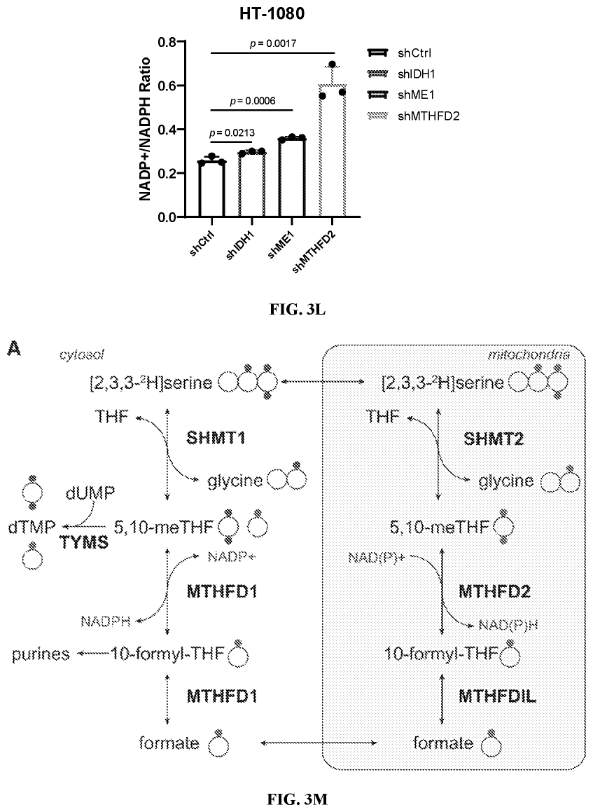

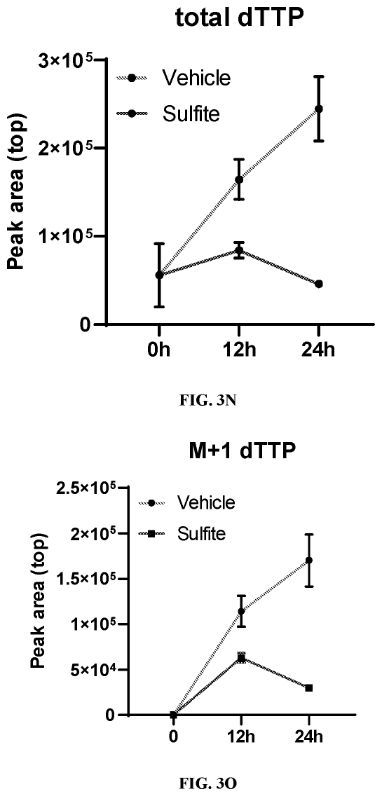

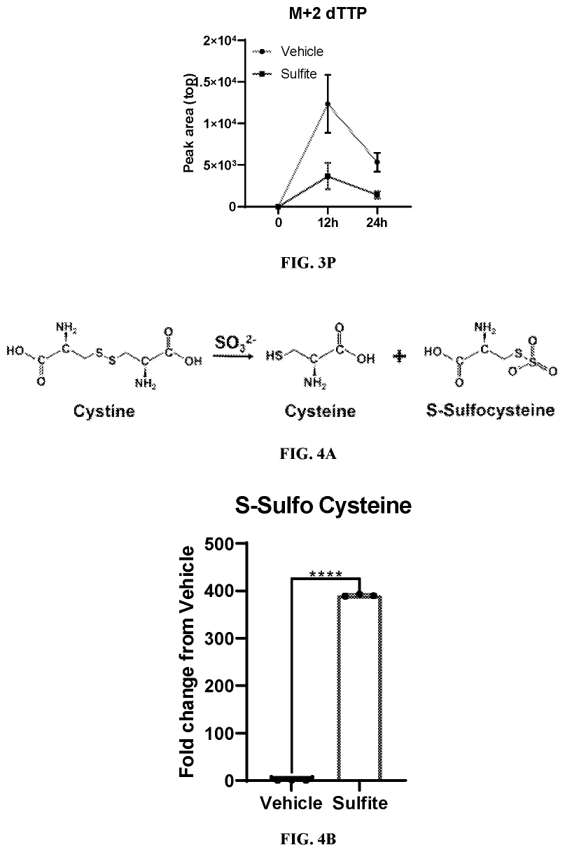

Metabolomic analysis was performed in vehicle or sulfite treated cells. The results are shown in FIG. 3A. changed metabolites induced by sulfite treatment enriched pathways are shown in FIG. 3B. An increase in the levels of 6-phospho-D-Gluconate (FIG. 3C), ribose phosphate (FIG. 3D), D-Glyceraldehyde 3-phosphate (FIG. 3E), D-sedoheptulose-1/7 phosphate (FIG. 3F), NDAP+/NADPH ratio (FIG. 3G), oxPPP shunting (FIG. 3H) were observed.

6-Aminonicotinamide (6-AN) is a competitive inhibitor of NADP+-dependent enzyme glucose-6-phosphate dehydrogenase (G6PD), 6-AN enhanced the cell death observed with RSL3. Similar to sulfite, 6-AN did not induce cell death on its own (FIG. 3I). As seen before, RSL3 and 6-AN in combination did not induce cell death when combined with Ferr-1. Similar results were observed when Epiandrosterone (EA) was used (FIG. 3J).

To generate knockdown cell lines, lentiviral transduction with IDH1/ME1/MTHFD2 shRNA vectors was conducted (FIG. 3K). NADP+/NADPH ratio was mostly increased in MTHFD2 knock down cell (FIG. 3L).

The pathway involving how serine is metabolized through one-carbon metabolism is shown in FIG. 3M.

The total dTTP, M+1dTTP and M+2 dTT were measured in the presence or absence of sulfite. As shown in FIG. 3N, FIG. 3O, FIG. 3P, the levels of all three decreased over time in sulfite treated samples compared to vehicle treated samples.

### Example 3: S-Sulfocysteine Sensitizes Cancer Cells to GPX4 Inhibition Induced Ferroptosis

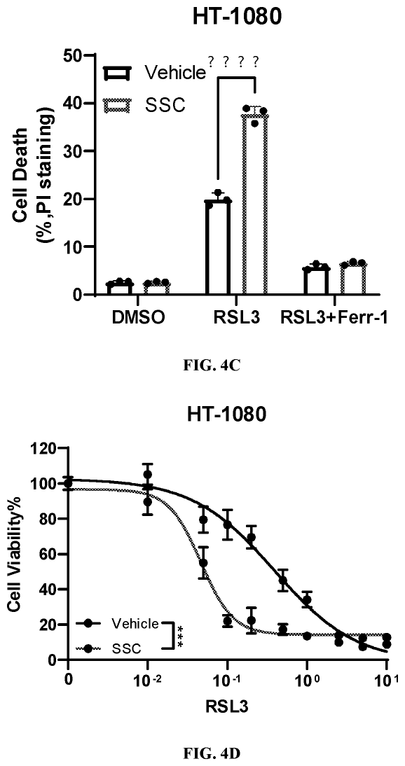

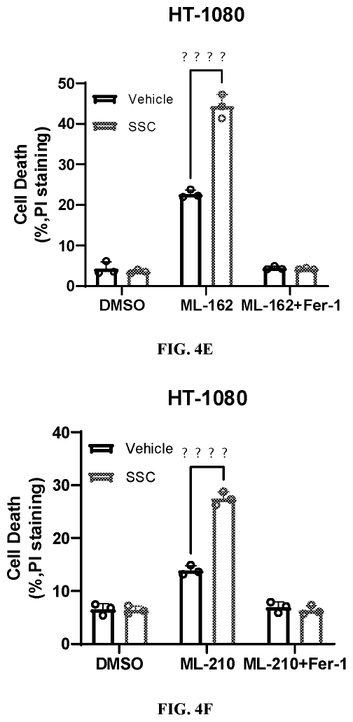

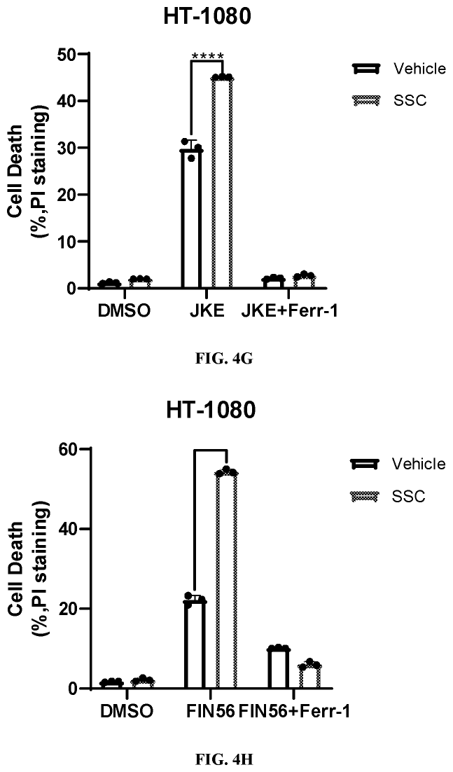

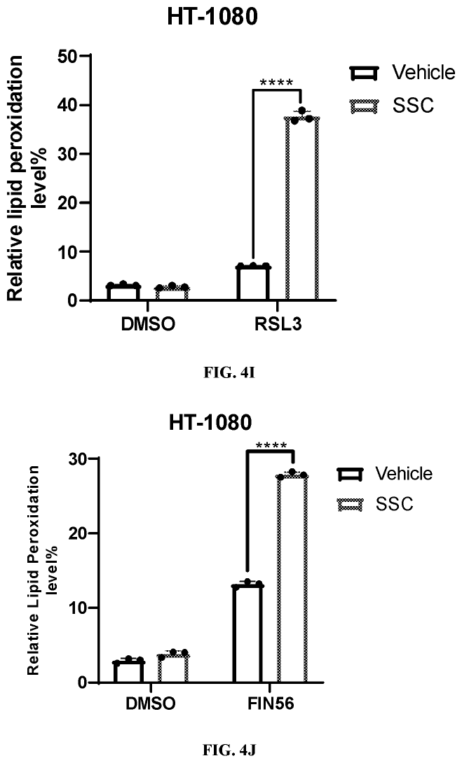

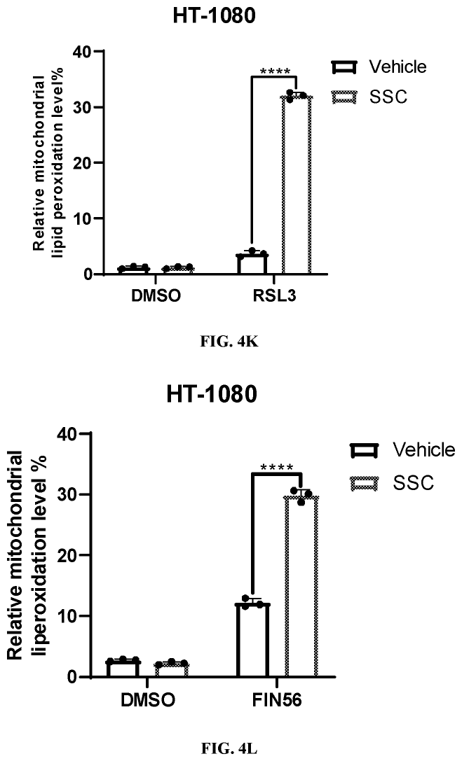

S-sulfocysteine is a product of sulfite and cysteine (FIG. 4A). Levels of S-sulfocysteine were found to increase in sulfite treated samples (FIG. 4B). Similar to sulfite, S-sulfocysteine also sensitized cancer cells to RSL3 (FIG. 4C) including over a range of RSL3 concentrations tested (FIG. 4D), ML-162 (FIG. 4E), ML-210 (FIG. 4F), JKE (FIG. 4G), FIN56 (FIG. 4H). Lipid peroxidation levels observed in RSL3 and S-sulfocysteine (FIG. 4I) or FIN56 and S-sulfocysteine (FIG. 4J) were increased compared RSL3 only or S-sulfocysteine only treated cells. Similar results were obtained for mitochondrial lipid peroxidation level (FIG. 4K and FIG. 4L).

S-sulfocysteine is produced through sulfite reaction with cystine. Reducing the cystine concentration in the media can reduce the production of S-sulfocysteine, which can rescue the cell death induced by sulfite plus RLS3, proving that sulfite function through producing S-sulfocysteine (FIG. 4M).

### Example 4: SHMT2 Inhibition Sensitizes Cancer Cells to GPX4 Inhibition Induced Ferroptosis

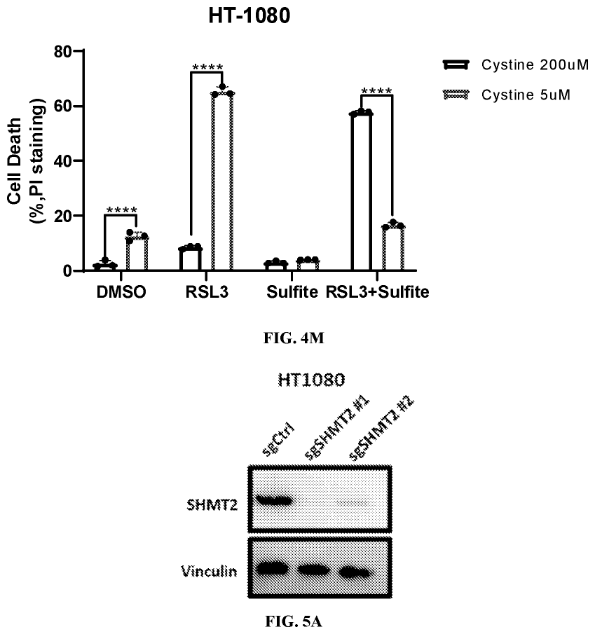

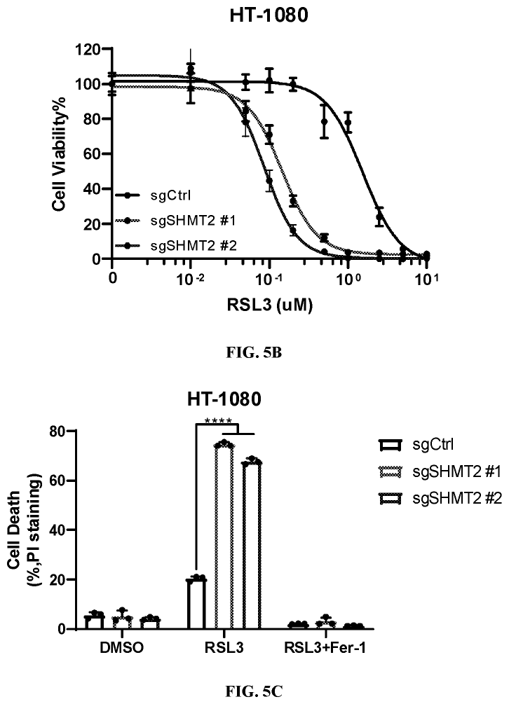

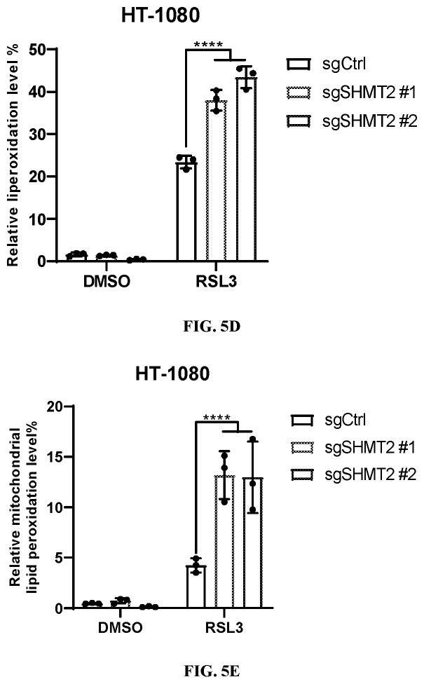

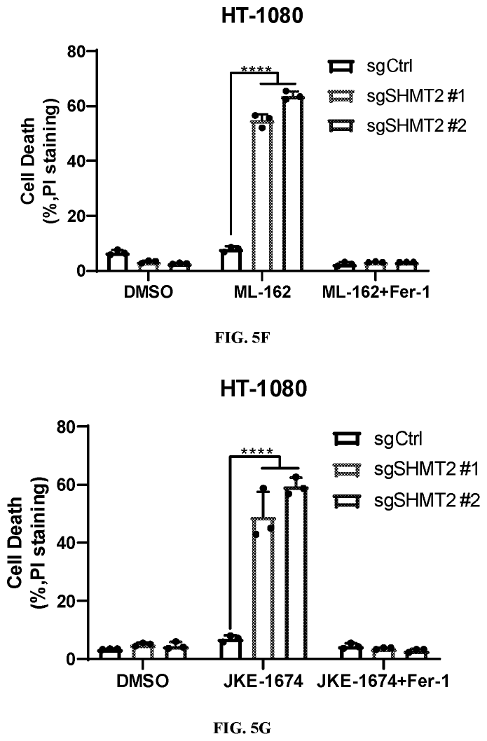

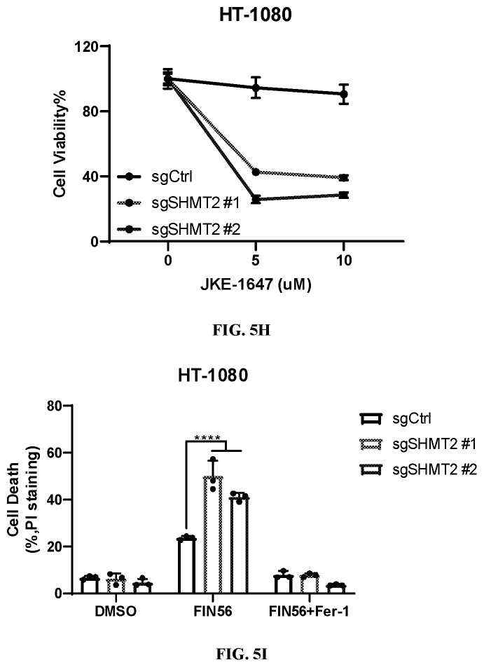

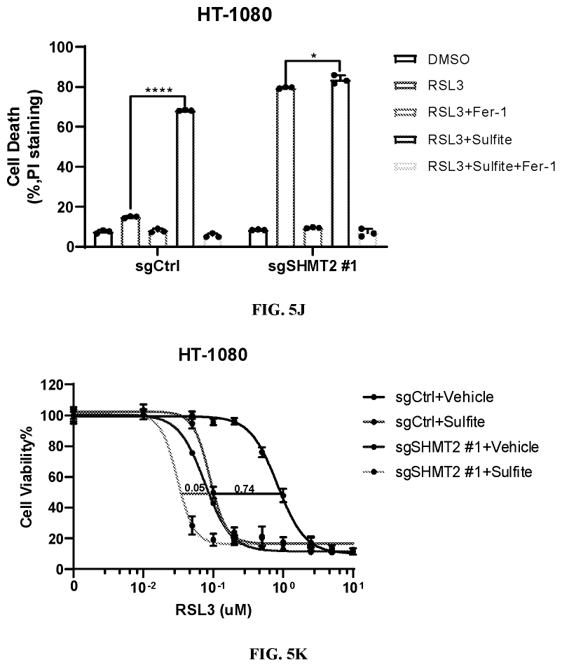

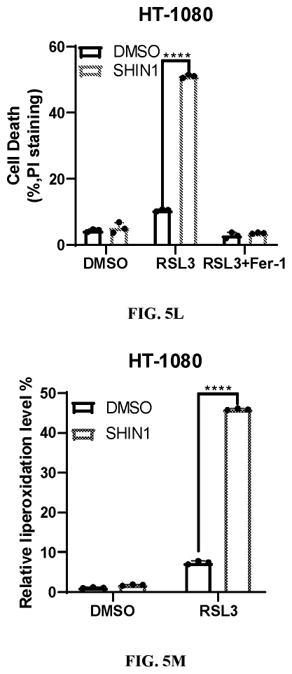

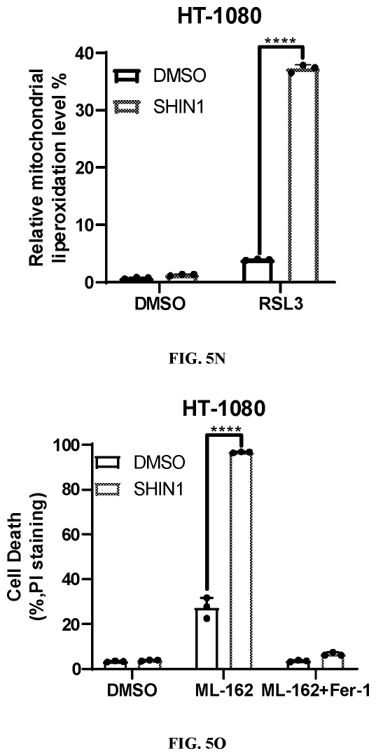

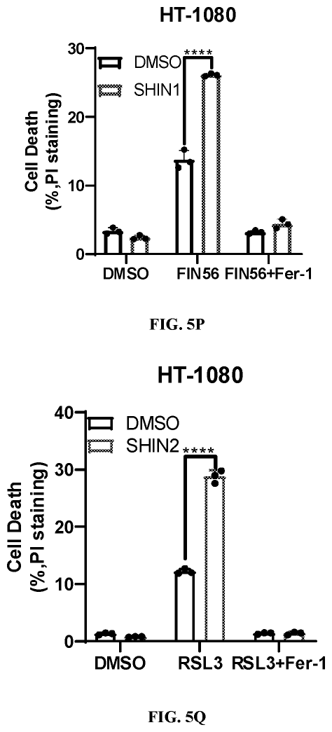

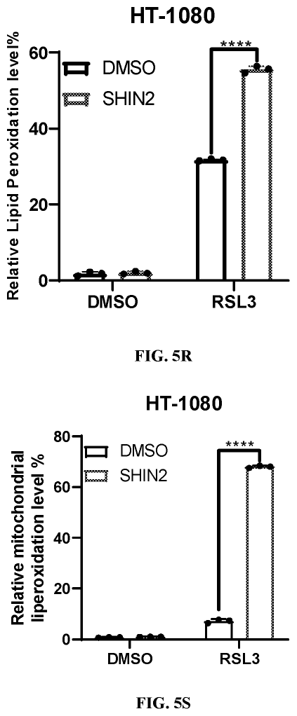

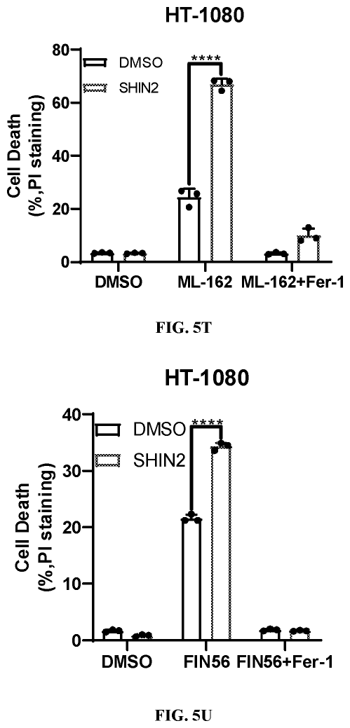

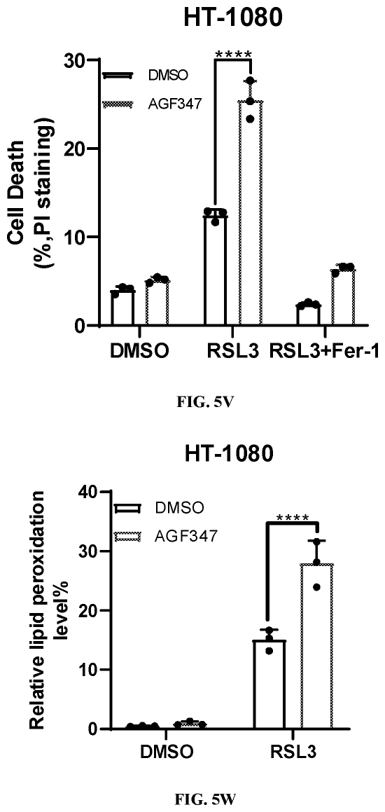

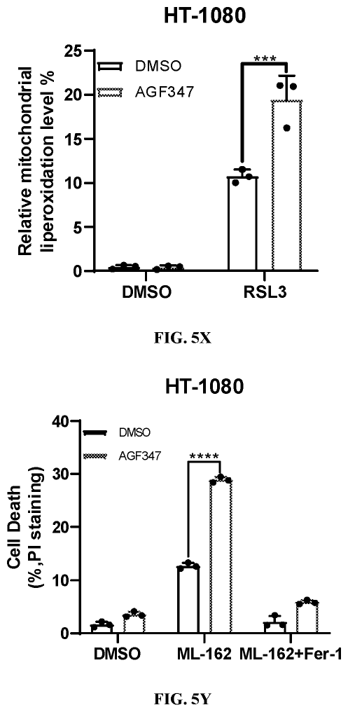

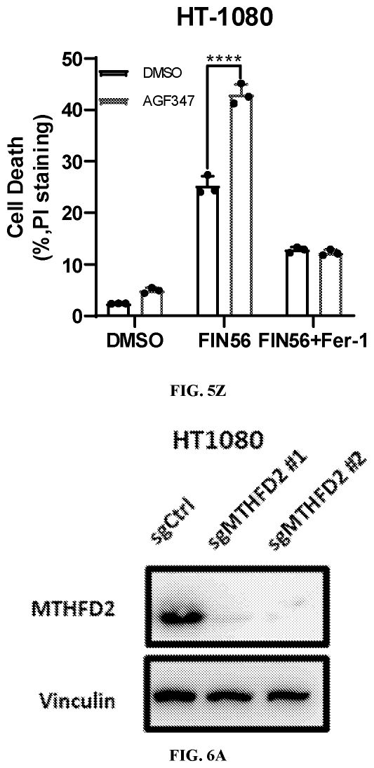

Knockout of SHMT2 was performed using CRISPR-Cas9 mediated gene knockout (FIG. 5A). Cell viability was reduced by SHTM2 knockout when combined with varying concentrations of RSL3 (FIG. 5B). Significantly, SHMT2 knockout increased RSL3 mediated cell death, which was not observed when combined with Fer-1 (FIG. 5C). As shown in FIG. 5D, increased lipid peroxidation level was observed in SHMT2 knock out cells upon RSL3 treatment. FIG. 5E shows the increased mitochondrial lipid peroxidation level in SHMT2 knock out cells upon RSL3 treatment.

Various ferroptosis inducers namely ML-162 (FIG. 5F), JKE-1674 (FIG. 5G and FIG. 5H), FIN56 (FIG. 5I) were able to, in SHMT2 knockout cells induced cell death and or decrease viability. SHMT2 knockout did not result in substantial cell death in these assays.

As shown in FIG. 5J, compared to RSL3 alone RSL3+Sulfite led to significant increased cell death in wild type cells. While RSL3+Sulfite only cause a slight increase in cell death. As shown in FIG. 5K, the sensitization effect of sulfite on SHMT2 knock out cells is much weaker than in wild type cells. Together FIGS. 5J and 5K show that sulfite functions through inhibiting SHMT2.

Small molecule SHMT inhibitors SHIN1, SHIN2, AGF347 were also tested in HT1080 cells. Similar to SHMT2 knockout, small molecule inhibitors of SHMT namely SHIN1, SHIN2, and AGF347, when combined with RSL3 increased cell death (FIG. 5L, FIG. 5Q, FIG. 5V), increased lipid peroxidation levels (FIG. 5M, FIG. 5R, FIG. 5W) and increased relative mitochondrial lipid peroxidation level (FIG. 5N, FIG. 5S, FIG. 5X). These results were also observed when SHIN1, SHIN2, and AGF347 were combined with ferroptosis inducers ML-162 (FIG. 5O, FIG. 5T, FIG. 5Y) or FIN56 (FIG. 5P, FIG. 5U, FIG. 5Z).

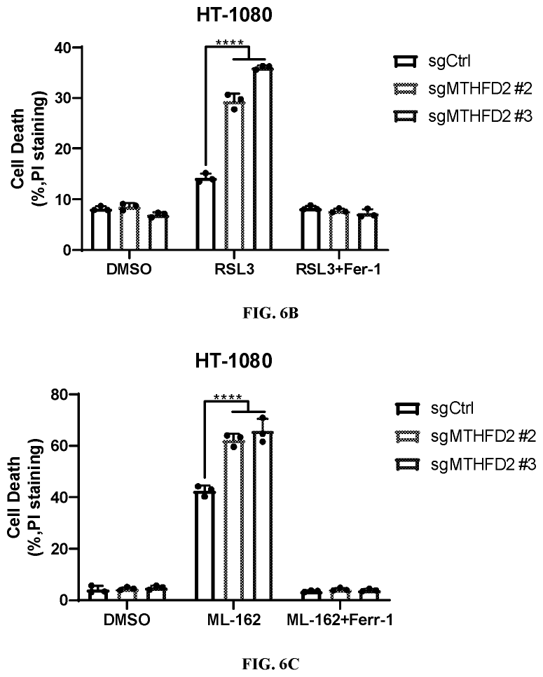

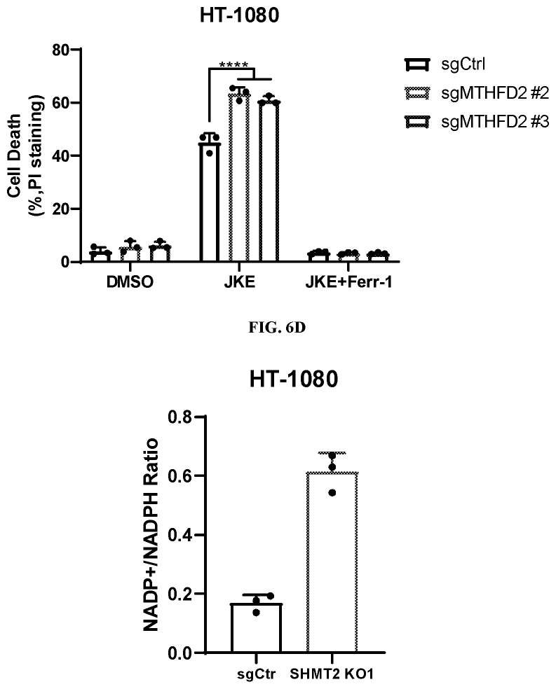

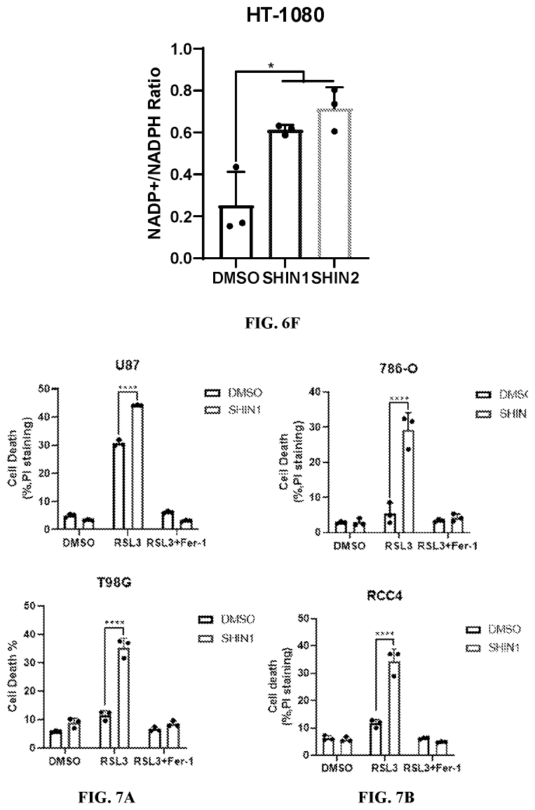

To test if disruption of one-carbon metabolism sensitizes cancer cells to GPX4 inhibition induced ferroptosis, MTHFD2 was knocked out in HT-1080 cells (FIG. 6A).

MTHFD2 knockout was able to significantly increase Ferroptosis inducers RSL3 (FIG. 6B), ML-162 (FIG. 6C), and JKE-1674 (FIG. 6D) mediated cell death. Significantly, MTHFD2 knockout did not induce cell death.

NADP+/NADPH ratios were also higher in SHMT2 knockout cells (FIG. 6E) and in cells treated with SHMT inhibitors SHIN1 and SHIN2 (FIG. 6F).

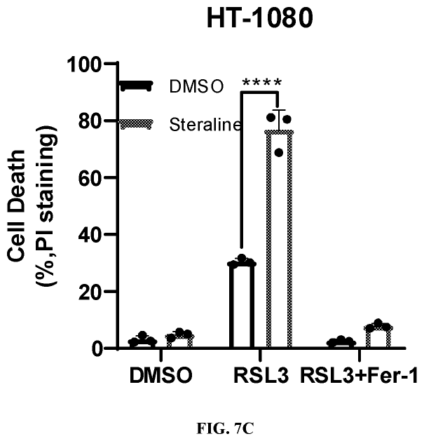

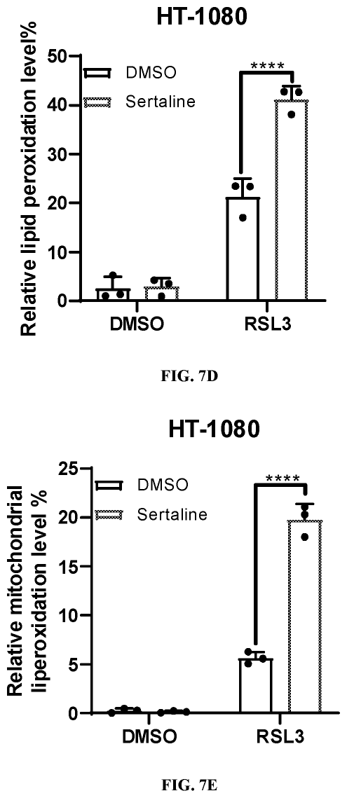

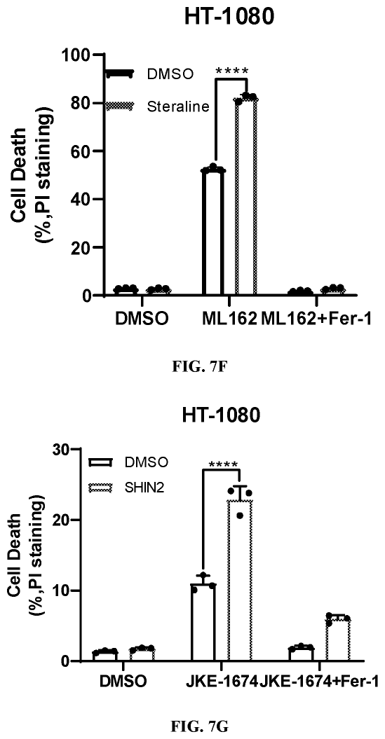

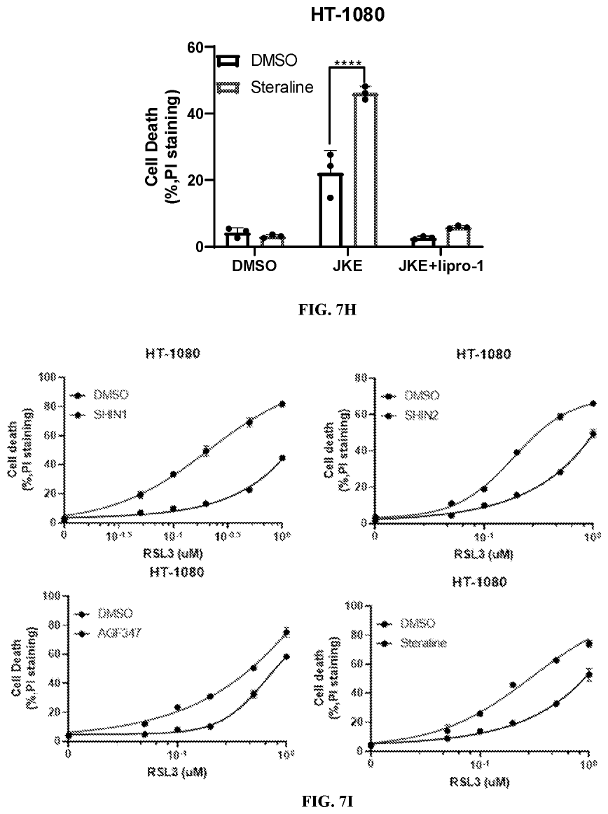

The ability of the SHMT inhibitors, SHIN1 and SHIN2 to enhance ferroptosis induced by RSL3 was observed in a panel of cell lines tested (FIG. 7A and FIG. 7B).

SHMT inhibitor sertraline was able to significantly increase in RSL3 mediated ferroptosis (FIG. 7C), lipid peroxidation (FIG. 7D) and mitochondrial lipid peroxidation (FIG. 7E). Sertraline was also able to significantly enhance the cell death observed with GPX4 inhibitor ML-162 (FIG. 7F).

SHIN2 and sertraline were also able to enhance cell death observed with ferroptosis inducer JKE-1674 (FIG. 7G and FIG. 7H).

SHIN1, SHIN2, AGF347, and sertraline were able to enhance cell death over various concentrations of RSL3 tested (FIG. 7I).

### Example 5: Sulfite Disrupts SHMT2 Tetramer Formation

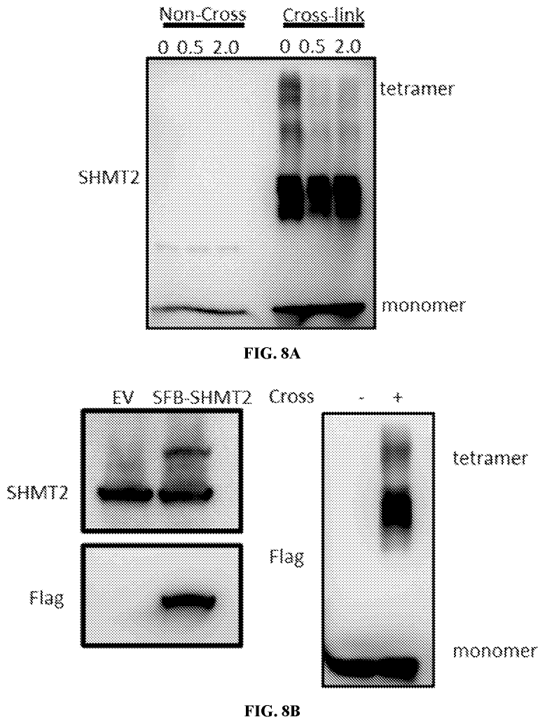

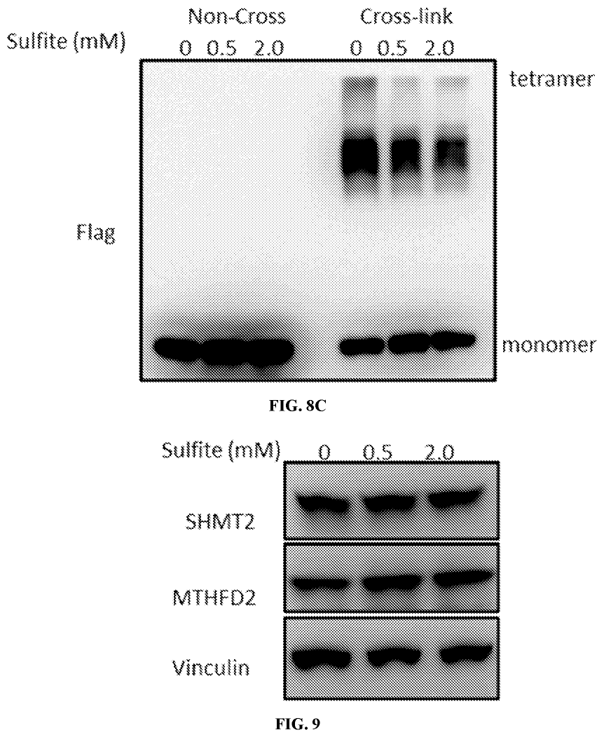

As shown in FIG. 8A, sulfite treatment was found to disrupt the tetramer formation of SHMT2 protein. Exogenously expressed SHMT2 was also able to form tetramer (FIG. 8B). Sulfite treatment also inhibited the tetramer formation of exogenous expressed SHMT2 (FIG. 8C).

These results indicate that sulfite treatment can inhibit the tetramer formation of SHMT2, which is important for SHMT2's function.

To test if sulfite changed mitochondrial protein levels, SHMT2 and MTHFD2 protein levels were measured following treatment with sulfite. No changes in protein levels were observed (FIG. 9).

### Example 6: Sulfite Sensitization Effect Did not Depend on Single Known Ferroptosis Regulator

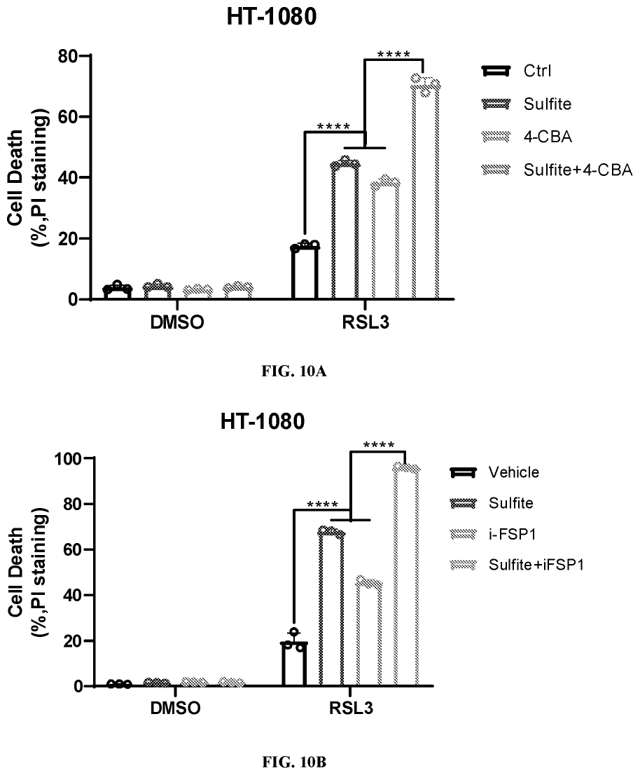

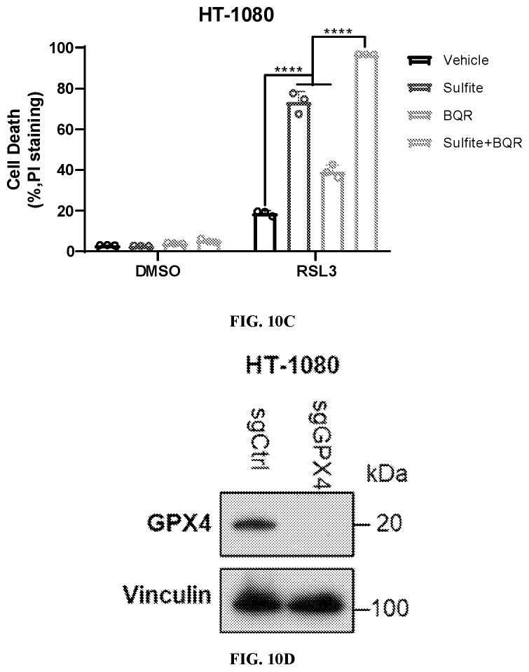

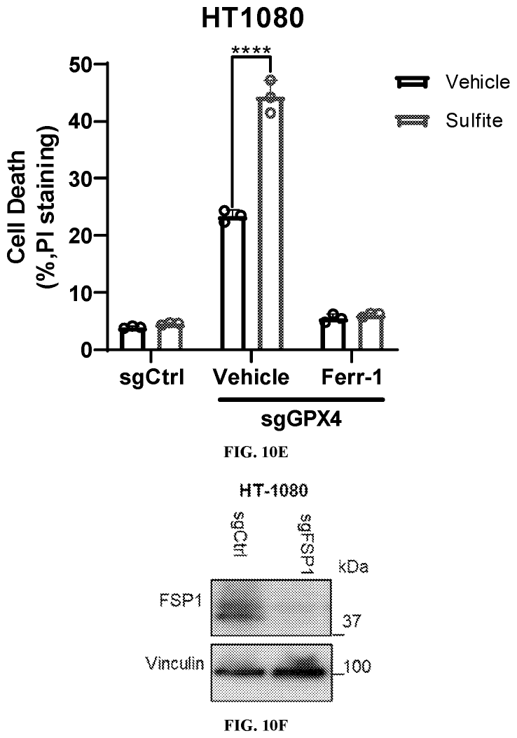

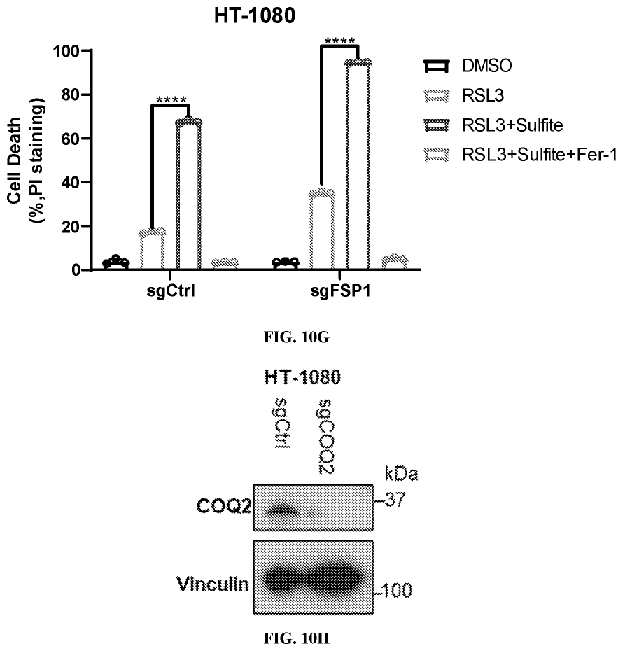

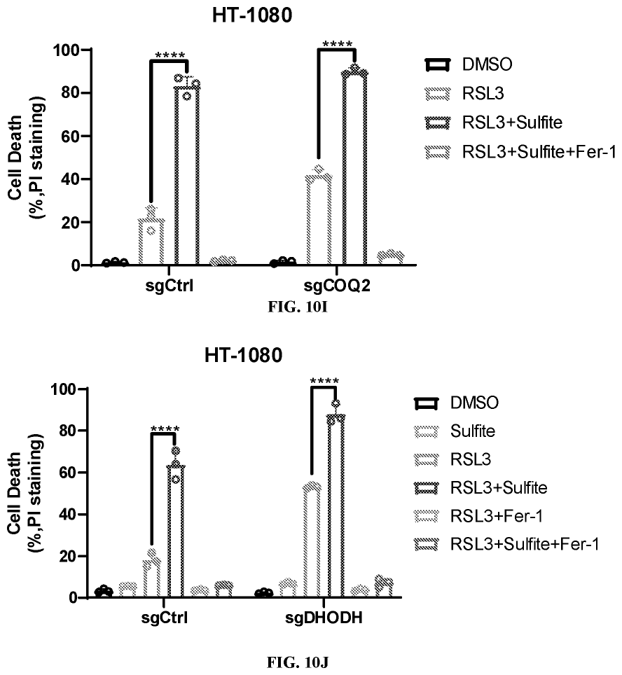

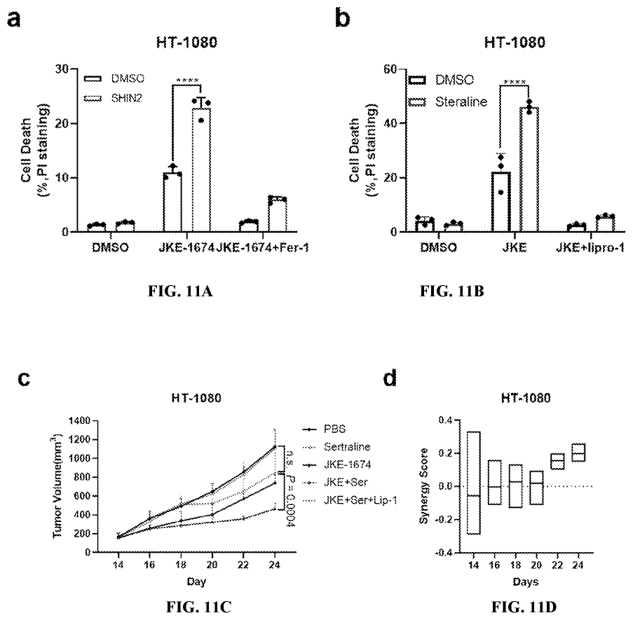

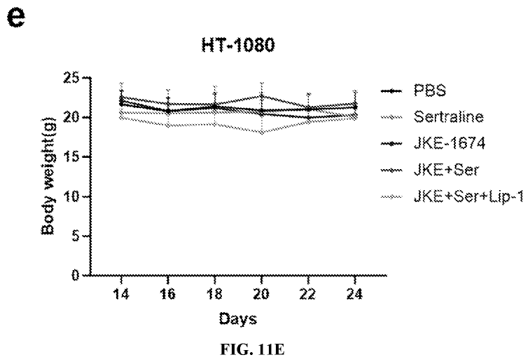

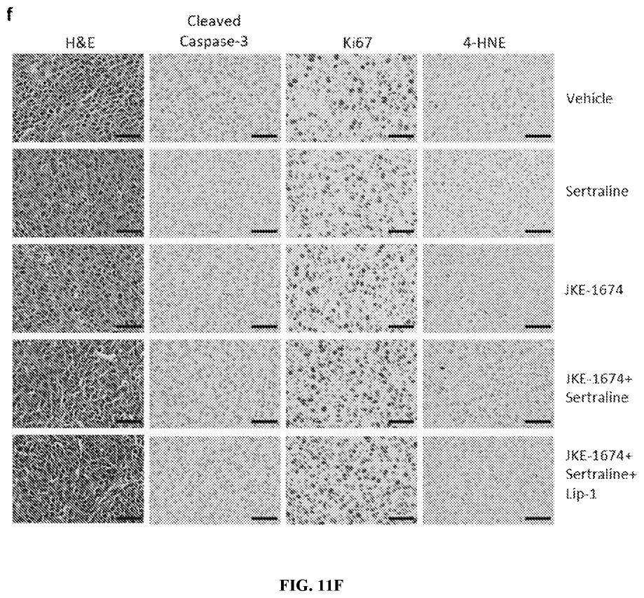

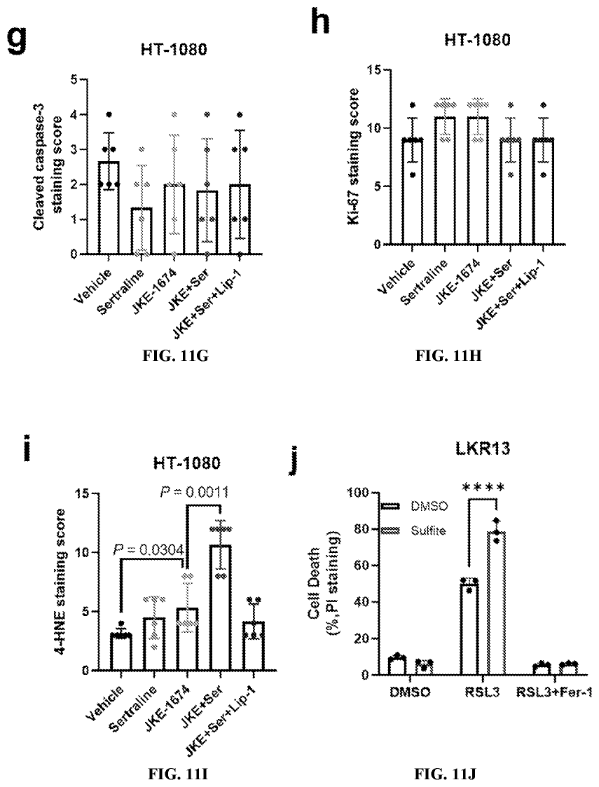

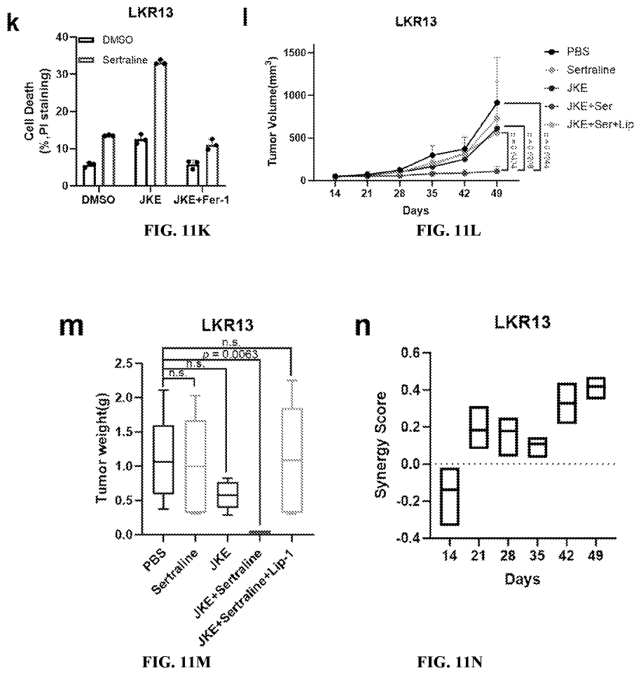

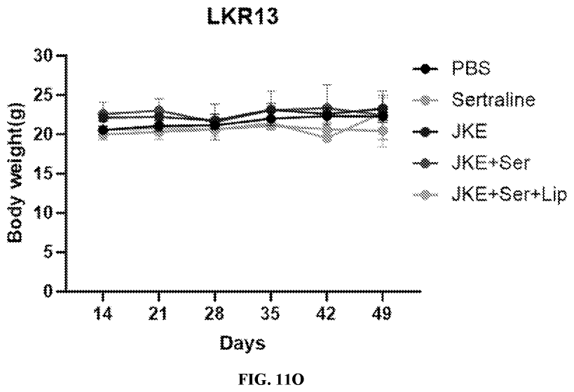

To test if sulfite sensitize cells to ferroptosis mainly through any known ferroptosis regulation axis CoQ biosynthesis inhibitor 4-Carboxybenzaldehyde (4CBA), FSP1 inhibitor iFSP1 and DHODH inhibitor brequinar (BQR) in co-treatment with RLS3 or RSL3 plus sulfite were used. Under these inhibitor treatments, sulfite can still sensitize cells to RSL3 induced ferroptosis (FIG. 10A, FIG. 10B, FIG. 10C).

To further prove this point, sulfite's sensitization effect on GPX4 knock out was further tested (FIG. 10D. FIG. 10E), FSP1 knock out (FIG. 10F, FIG. 10G), COQ2 knock out (FIG. 10H-I) and DHODH knock out (FIG. 10J) cell lines. These experiments showed that sulfite can still sensitize these knock out cells to ferroptosis.

### Example 7: Effect of SHMT Inhibitor and GPX4 Inhibitors on Tumor Growth In Vivo

To test the combination of SHMT inhibitors and GPX4 inhibitors on tumor growth, HT-1080 cells are implanted into athymic nude mice in a xenograft assay. Patient-derived xenograft (PDX) model is also used in parallel experiments. Mice are treated with varying concentrations of (i) SHIN2 (ii) sertraline (iii) JKE-1674 (iv) JKE-1674 and SHIN2 (v) JKE-1674 and sertraline (vi) JKE-1674, SHIN2 and Lipro-1 (vii) JKE-1674, sertraline and Lipro-1 as well as vehicle. Tumor volume and mouse weight are monitored over time. At the end of the assay, the mice are euthanized, and the tumors are harvested. Tumor weight is measured. Tissue section of the tumor are stained for Ki-67 and 4-HNE. The combination of JKE-1674 and SHIN2 as well as JKE-1674 and sertraline are expected to decrease tumor volume and weight compared to JKE-1674 alone or SHIN2 or sertraline alone.

### Example 8: Effect of SHMT Inhibitor and GPX4 Inhibitors on Tumor Growth In Vivo

To assess the in vivo impact of the combination of SHMT inhibition and GPX4 inhibition, the SHMT inhibitor sertraline was utilized, an FDA-approved drug, and the GPX4 inhibitor JKE-1674 for both in vitro and in vivo cell treatments. The data reveal that both SHIN2 and sertraline sensitize cancer cells to JKE-1674-induced ferroptosis (FIG. 11A-11B). Moreover, in vivo experiments with nude mice demonstrated that the combination of sertraline and JKE-1674 significantly impedes the growth of HT-1080-derived xenografts, whereas individual treatments showed no discernible effect (FIG. 11C). Synergy score calculations further confirmed the synergistic effect of sertraline and JKE-1674 in treating cancer cells (FIG. 11D). Notably, there was no significant difference in the weight of mice among different treatment groups (FIG. 5E). While sertraline alone or in combination with JKE-1674 did not alter staining for cleaved caspase-3 or Ki-67 in these tumors, the combination markedly increased staining for 4-HNE, a lipid peroxidation marker, in comparison to single treatments (FIG. 11F-I). Additionally, liproxstain-1 reversed the 4-HNE staining score back to levels similar to the vehicle control (FIG. 11F-I).

The assessment of ferroptosis function on the growth of either cell-derived or patient-derived xenografts is typically conducted in immunodeficient mice, such as nude mice or NSG mice. However, for applications in clinical settings, it becomes important to evaluate ferroptosis function in immunocompetent mice. To gain a more comprehensive understanding of the role of SHMT2 inhibition in combination with ferroptosis in cancer treatment, the investigations were extended to LKR13, a mouse cell line, both in vitro and in vivo. Initially, it was confirmed that sertraline promotes JKE-1674-induced ferroptosis in vitro (FIG. 11J-11K). Subsequently, the effects of sertraline, JKE-1674, and their combination were examined on a LKR-13 tumor growth in a 129/Sv mouse model. Similar to the findings in the HT-1080 model, sertraline or JKE-1674 treatment alone did not impact LKR13 tumor graft growth. However, the combination of sertraline and JKE-1674 significantly inhibited tumor growth (FIG. 11I). Furthermore, the combination significantly reduced tumor weight compared to other treatment groups, with no observable effect on mice weight (FIG. 11M-N). Synergy score calculations supported the synergistic effect between sertraline and JKE-1674 (FIG. 11O).

### EXPERIMENTAL METHODS

### Cell Culture Studies

HT-1080, 786-O, U87, T98G, RCC4, H1299, A549, Hela and Hs578T cancer cell lines were obtained from ATCC. All cell lines used in this study were free of mycoplasma contamination (per testing done by the vendor). All the cells were cultured in a 37° C. incubator in a 5% CO₂ atmosphere. H1299 cells were cultured in RPMI-1640 medium supplemented with 10% fetal bovine serum and 10,000 U/mL penicillin-streptomycin. HT-1080, 786-O, U87, T98G, RCC4, A549, Hela and Hs578T cells were cultured in Dulbecco modified Eagle medium (DMEM) supplemented with 10% fetal bovine serum and 10,000 U/mL of penicillin-streptomycin.

### Reagents

1S, 3R-RSL3 (#6118) and Z-VAD-FMK (#FMK001) were purchased from R&D systems. ML-162 (#20455), ML-210 (#23282), FIN56 (#25180), MitoTEMPO (hydrate) (#16621) and Liproxstatin-1 (#17730) were purchased from Cayman Chemical. JKE-1674 (#HY-138153) and SHIN1 (#HY-112066) were purchased from MedChemExpress. L-SERINE (2,3,3-D3) (#NC0285211) was purchased from Fisher. SHIN2 (#AOB11286) was purchased from AOBIOUS. Deferoxamine mesylate (#S5742), Necrostatin-1 (#S8037) and Epiandrosterone (#S2832) were purchased from Selleck. The following reagents were obtained from Sigma-Aldrich: sodium sulfite (#S4672), sodium bisulfite (#243973), 4-Hydroxy-TEMPO (#176141), Ferrostatin-1 (#SML0583), Sodium sulfate (#239313), Sodium Thiosulfate (#217263), 6-Aminonicotinamide (#A68203), L-Cysteine S-sulfate (#C2196), AGF347 (#SML2904) and sertraline hydrochloride (#S6319). All reagents were dissolved following the manufacturers' instructions.

### CRISPR-Cas9-Mediated Gene Knockout

Knockout of SUOX, SHMT2 and MTHFD2 in human cell lines were performed using single guide RNAs (sgRNAs) as described (Lee, H. et al. Nat Cell Biol 22, 225-234, doi: 10.1038/s41556-020-0461-8 (2020)). sgRNAs were cloned into the LentiCRISPR-V2 (Addgene, #52961) according to the standard protocol, as described previously (see Wu, S. et al. Proc Natl Acad Sci USA 119, e2121987119, doi: 10.1073/pnas.2121987119 (2022); Mao, C. et al. Nature 593, 586-590, doi: 10.1038/s41586-021-03539-7 (2021)). The knockout cells were identified by immunoblotting. The sequences of the single-guide RNAs are listed in Table 1 below:

TABLE 1
Single-guide RNA sequences

| sgRNA name | sgRNA sequence | SEQ ID |
|---|---|---|
| sgCtrl | GGCACTACCAGAGCTAACTCA | 1 |
| sgSUOX #2 | GCATCTCAGAGCGTCGGTTGC | 2 |
| sgSUOX #4 | GGTGACTGTGATCTCGTACCT | 3 |
| sgSHMT2 #1 | GAGAAGGACAGGCAGTGTCG | 4 |
| sgSHMT2 #2 | GTAGACGGCCAGGTTGGCTG | 5 |
| sgMTHFD2 #1 | GAGGGAGTGGAACCTCGATAT | 6 |
| sgMTHFD2 #2 | GCGCCAACCAGGATCACACTC | 7 |
| sgGPX4 | GGGTGAAGCGCTACGGACCCA | 8 |
| sgCOQ2 | ATGCTGGGCTCGCGAGCCGC | 9 |
| sgFSP1 | TCCCGATTCCACCGAGACCT | 10 |

LentiGuide clones were transfected into HEK293T cells with psPAX2 (packaging plasmid) and pMD2.G (expressing plasmid). Cells were infected with lentivirus with 8 μg/ml polybrene and selected with puromycin (2 μg/ml, InvivoGen) for 2 days, and then single cells were sorted into 96-well plates. Single cells were maintained in DMEM with 10% (v/v) FBS and 1% (v/v) penicillin/streptomycin at 37° C. in an incubator with 20% 02 and 5% CO₂ for 3-4 weeks and each colony was verified by immunoblotting to confirm target gene deletion.

### Overexpression or Knock Down Cell Line Generation

Cell lines with stable overexpression of target genes were generated as previously described (Chauhan, A. S. et al. FASEB J 33, 2957-2970, doi: 10.1096/fj.201801225R (2019); Zhang, Y., et al. Cell Cycle 18, 773-783, doi: 10.1080/15384101.2019.1597506 (2019)). To generate cell lines with stable overexpression of C-terminal-tagged SHMT2, the SHMT2 cDNA was cloned into the lentivirus expression vector pLEX_307 SFB. HEK293T cells were transfected with either pLEX_307 SFB-empty vector, or -SHMT2, together with the lentiviral packaging plasmids psPAX2 and pMD2.G using Lipofectamine 2000 reagent (Life Technologies, #11668030) according to the manufacturer's instructions and as described previously (Lei, G. et al. Oncogene 40, 3533-3547, doi: 10.1038/s41388-021-01790-w (2021); Lei, G. et al. Cell Res 30, 146-162, doi: 10.1038/s41422-019-0263-3 (2020)). Forty-eight hours later, lentivirus was collected and filtered, and the target cell lines were infected with lentivirus. Twenty-four hours after infection, antibiotic was added to the medium to obtain stable cell lines. Protein expression levels were determined by immunoblotting. To generate knockdown cell lines, lentiviral transduction with IDH1/ME1/MTHFD21 shRNA vectors was conducted as described previously (Lin, A. et al. Cancer Res 74, 1682-1693, doi: 10.1158/0008-5472.CAN-13-1729 (2014)). Forty-eight hours later, upon puromycin antibiotic selection, protein expression levels were determined by immunoblotting.

### Cell Death Assay

Cell death was measured and analyzed using flow cytometry as described previously (Zhang, Y., et al. Cell Cycle 18, 773-783, doi: 10.1080/15384101.2019.1597506 (2019); Koppula, P. et al. Nature communications 13, 2206, doi: 10.1038/s41467-022-29905-1 (2022)). Briefly, cells were seeded in 12-well plates and incubated overnight. After treatment, cells were trypsinized and collected. After washing with cold phosphate-buffered saline (PBS), the cells were stained with 2 μg/mL propidium iodide (PI) in cold PBS. The fraction of dead cells (PI-positive cells) was measured using a Attune Flow Cytometer (ThermoFisher) and analyzed by FlowJo 10 software (Lin, A. et al. Oncogene 33, 3183-3194, doi: 10.1038/one.2013.273 (2014); Liu, X. & Gan, B. et al. Cell Cycle 15, 3471-3481, doi: 10.1080/15384101.2016.1249545 (2016)). All experiments were performed in triplicates.

### Lipid Peroxidation Measurement

Cells were seeded on 12-well plates and incubated overnight. After treating with compounds for indicated time, cells were incubated in media containing 5 μM C11-BODIPY 581/591 (Invitrogen) for 30 min at 37° C. Then cells were trypsinized and collected. After washing with cold phosphate-buffered saline (PBS), cells were resuspended in PBS. Then lipid peroxidation was measured using a Attune Flow Cytometer (ThermoFisher) with a 488 nm laser and analyzed by FlowJo 10 software. All experiments were performed in triplicates.

### Cell Viability Assay

Cell viability was measured using a Cell Counting Kit-8 (CCK-8, Dojindo) as previously described (Koppula, P., et al. J Biol Chem 292, 14240-14249, doi: 10.1074/jbc.M117.798405 (2017); Liu, X. et al. Nat Cell Biol 18, 431-442, doi: 10.1038/ncb3328 (2016)). In brief, cells were seeded onto 96-well plates at a density of 1×10⁴ per well. The next day, cells were treated with indicated compound for 16-20 h. Subsequently, cells were changed to new 100 μl medium containing 10 μl CCK-8 reagent for 1 h at 37° C., 5% CO₂ in incubator. The absorbance at 450 nm was determined using a FLUOstar Omega microplate reader (BMG Labtech).

### NADP+ and NADPH Measurement

The intracellular levels of NADPH and total NADP (NADPH+NADP+) were measured as per Liu, X. et al. Nat Cell Biol 22, 476-486, doi: 10.1038/s41556-020-0496-x (2020). Briefly, cells were seeded in 6-well plates and incubated overnight. After treatment, cells were washed with cold phosphate-buffered saline (PBS) and lysed in 300 μL extraction buffer (20 mM nicotinamide, 20 mM NaHCO₃, 100 mM Na₂CO₃) and centrifuged at 15,000 rpm for 10 min. The supernatant was split into two 150-μL aliquots. For the measurement of total NADP, 20 μL supernatant from one 150 μL aliquot and 80 μL of NADP-cycling buffer (100 mM Tris-HCl [pH8.0], 0.5 mM thiazolyl blue, 2 mM phenazine ethosulfate, 5 mM ethylenediaminetetraacetic acid [EDTA]) containing 1.0 U of G6PD enzyme (Sigma-Aldrich, #G4134) were mixed into a 96-well plate. After incubation for 1 minute in the dark at 30° C., 20 μL of 10 mM fresh glucose 6-phosphate solution was added to the mixture, and the change of absorbance at 570 nm was measured with a microplate reader every 30 seconds for 5 minutes at 30° C. For the NADPH measurement, the remaining 150 μL supernatant was incubated at 60° C. for 30 minutes (to destroy NADP⁺ without affecting NADPH), followed by the same procedures as those performed for the measurement of total NADP. Eventually, the concentration of NADP⁺ was calculated by subtracting [NADPH] from [total NADP]. All experiments were performed in triplicate.

### Western Blotting

Briefly, cells were lysed in NP-40 lysis buffer (50 mM Tris [pH 7.4], 250 mM NaCl, 5 mM EDTA, 50 mM NaF, 1 mM Na₃VO₄, 1% Nonidet P40) containing complete mini protease inhibitors (Roche). The protein concentration of lysates was quantified using BCA Protein Assay Kit from Life Technologies (#23227). 20 μg of total protein was loaded into SDS-PAGE gel and transferred to a PVDF membrane (Bio-Rad) using standard techniques. The primary antibodies and concentrations used for Western blotting were Vinculin (1:1000, Sigma-Aldrich, #V4505), SUOX (1:1000, Abcam, #ab129094), SHMT2 (1:1000, Sigma-Aldrich, #HPA-020549), IDH1 (1:1000, Proteintech, #12332-1-AP), ME1 (1:1000,), MTHFD2 (1:1000, Abcam, #ab56772), Flag (1:1000, Sigma, #F1804), GPX4 (1:1,000, R&D, #MAB5457), COQ2 (1:1,000, Santa Cruz, sc-517107) and FSP1 (1:1000, Proteintech, #20886-1-AP).

### Metabolomic Analysis

Metabolomic analysis was performed as previously described (Liu, X. et al. Nat Cell Biol 22, 476-486, doi: 10.1038/s41556-020-0496-x (2020)). Cells were seeded on 35-mm culture plates at a density sufficient to ensure approximately 70-80% confluence at the time of extraction. Metabolites were extracted at the indicated time points for subsequent analyses. Metabolites were extracted by rapidly aspirating the culture medium and then adding 500 μl of an 80% methanol: 20% water mixture at dry ice temperature. The plates were incubated on dry ice for 15 min before the cells were scraped into pre-chilled Eppendorf tubes. The cell debris was pelleted by centrifugation at 13,000 ref for 5 min at 4° C. and the supernatant was transferred into a fresh tube and stored on dry ice until analysis. Just before analysis, 500 μl of extract was dried under nitrogen gas flow and then resuspended in 100 μl water.

The complete platform consists of an Accela 1250 HPLC system, Accela Open Autosampler, MayLab Mistraswitch column oven and Exactive orbitrap mass spectrometer, controlled by the Xcalibur 3.0.63 software package. Chromatography was performed with a Phenomenex Synergi Hydro-RP column (100×2 mm, 2.5 μm particle size). Solvent A is 10 mM tributylamine and 15 mm acetic acid in water; Solvent B is methanol. The gradient is: 0 min, 0% B; 2.5 min, 0% B; 5 min, 20% B; 7.5 min, 20% B; 13 min, 55% B; 15.5 min, 95% B; 18.5 min, 95% B; 19 min, 0% B; 25 min, 0% B. The injection volume was 10 μl. The column temperature was set to 40° C., and the flow rate was 200 μl/min. The Exactive was operated in negative ionization mode with an electrospray ionization interface. The instrument parameters are as follows: sheath gas flow rate 30 (arbitrary units), aux gas flow rate 10 (arbitrary units), sweep gas flow rate 3 (arbitrary units), spray voltage 3 kV, capillary temperature 325° C., capillary voltage −25 V, tube lens voltage −50 V. The scan range was set to 80-1,000 m/z, with a maximum inject time of 250 ms, resolution of 100,000 at 1 Hz, and AGC (automatic gain control) target 1×10⁶. The data were analyzed using the MAVEN software suite with signal intensity determined as the Peak Area (Top). For metabolic labelling experiments, the data were corrected to account for the natural abundance of nitrogen-15 using IsoCorrectoR (Heinrich, P. et al. Sci Rep 8, 17910, doi: 10.1038/s41598-018-36293-4 (2018)).

### Oxidative PPP Flux Calculations

Cells were seeded in six-well plates and incubated overnight, then given 2 ml of fresh medium containing 25 mM 1,2-[13C2] glucose. Aliquots of this medium were frozen and stored for analysis to determine the initial glucose concentration, and cells in replicate plates were trypsinized and counted to determine initial cell numbers. After 24 h, the spent medium was extracted as above and frozen until analysis, and the cells were trypsinized and counted to determine the final cell numbers.

For lactate isotopomer analysis the samples were analyzed directly. The relative quantities of M0, M1, M2 and M3 lactate were corrected for natural carbon-13 abundance using the IsoCorrectoR software package (Heinrich, P. et al. Sci Rep 8, 17910, doi: 10.1038/s41598-018-36293-4 (2018)). and the oxidative PPP-shunting ratio was calculated by taking the ratio M1/(M1+M2) (Badur, M. G. et al. Cell Rep 25, 1680, doi: 10.1016/j.celrep.2018.10.099 (2018)); since there is no difference in growth rates between the vehicle and sulfite treated cell lines, it is assumed that the biosynthetic demand for ribose-5-phosphate or any other PPP intermediate is the same in each case, so any increase in this ratio reflects an increase in NADPH production by the oxidative PPP.

### Statistics and Reproducibility

The results of all cell culture experiments were collected from at least three independent replicates. Data are presented as means+standard deviation (s.d.). Statistical significance (P values) was calculated using unpaired Student's t-tests or log-rank test by GraphPad Prism 8.0. *P<0.05; **P<0.01; ***P<0.001; ****P<0.0001; NS, not significant. No statistical methods were used to predetermine sample size. The investigators were not blinded to allocation during experiments and outcome assessment.

Although the invention has been described with reference to the presently preferred embodiment, it should be understood that various modifications can be made without departing from the spirit of the invention. Accordingly, the invention is limited only by the following claims.

What is claimed is:

1. A method of treating cancer in a subject comprising administering to the subject:
a) one or more one-carbon metabolism inhibitors; and
b) one or more ferroptosis inducers;
wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby treating cancer in the subject.

2. A method of enhancing ferroptosis in a tumor comprising exposing the tumor to:
(a) one or more one-carbon metabolism inhibitors; and
(b) one or more ferroptosis inducers,
wherein the ferroptosis induced in the tumor is greater than the ferroptosis induced when the tumor is exposed to one or more ferroptosis inducers or one or more one-carbon metabolism inhibitors alone, and
wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby enhancing ferroptosis in the tumor.

3. A method of sensitizing cancer cells to ferroptosis comprising exposing the cancer cells to one or more one-carbon metabolism inhibitors,
wherein at least one one-carbon metabolism inhibitor is a serine hydroxymethyltransferase (SHMT) inhibitor, a methylenetetrahydrofolate dehydrogenase (MTHFD) inhibitor or a combination thereof, thereby sensitizing the cancer cells to ferroptosis.

4. The method of any one of claims 1-3, wherein ferroptosis is induced by administering one or more ferroptosis inducers.

5. The method of any one of claims 1-4, wherein at least one ferroptosis inducer is a glutathione peroxidase 4 (GPX4) inhibitor.

6. The method of any one of claims 1-4, wherein the SHMT inhibitor is an SHMT1 inhibitor, an SHMT2 inhibitor or an SHTM1/SHMT2 inhibitor.

7. The method of any one of claims 1-4, wherein the MTHFD inhibitor is a MTHFD1 inhibitor, MTHFD2 inhibitor, an MTHFD1L inhibitor or a combination thereof.

8. The method of any one of claims 1-4, wherein the one or more one-carbon metabolism inhibitors and the one or more ferroptosis inducers are administered at the same time.

9. The method of any one of claims 1-4, wherein the one or more one-carbon metabolism inhibitors and the one or more ferroptosis inducers are administered sequentially.

10. The method of any one of claims 1-4, wherein at least one SHMT inhibitor is SHIN2, AGF347, SHIN1, sertraline, Compound 2.12, Compound 2.2, sulfite, S-sulfocysteine, or a combination thereof.

11. The method of claim 5, wherein at least one GPX4 inhibitors is RSL3, FIN56, FINO₂, ML-210, M-162, JKE 1674 or a combination thereof.

12. The method of any one of claims 1-4, wherein administering the one or more one-carbon metabolism inhibitors and the one or more ferroptosis inducers results in an increase in ferroptosis.

13. The method of claim 1, wherein the cancer is selected from leukemia, breast cancer, kidney cancer, brain cancer, non-small cell lung cancer, prostate cancer, colon cancer, cervical cancer, or fibrosarcoma.

14. The method of any one of claims 1-13, wherein an effective dosage of at least one of the SHMT inhibitor, the MTHFD inhibitor, and the GPX4 inhibitor is administered to a subject in need thereof.

15. The method of claim 14, wherein the effective dosage of the SHMT inhibitor is about 0.0025 to about 1500 mg/kg, the effective dosage of the MTHFD inhibitor is about 0.0025 to about 1500 mg/kg, and the effective dosage of the GPX4 inhibitor is about 0.0025 to about 1500 mg/kg.

16. A pharmaceutical composition comprising:
a) one or more one-carbon metabolism inhibitors;
b) one or more ferroptosis inducers; and
c) a pharmaceutically acceptable carrier.

17. The pharmaceutical composition of claim 16, wherein at least one one-carbon metabolism inhibitor is a serine hydroxymethyltransferase (SHMT) inhibitor, a methylenetetrahydrofolate dehydrogenase (MTHFD) inhibitor or a combination thereof.

18. The pharmaceutical composition of claim 16-17, wherein the SHMT inhibitor is an SHMT1 inhibitor, an SHMT2 inhibitor or an SHTM1/SHMT2 inhibitor.

19. The pharmaceutical composition of claim 16-18, wherein the SHMT inhibitor is SHIN2, AGF347, SHIN1, sertraline, Compound 2.12, Compound 2.2, sulfite, S-sulfocysteine, or a combination thereof.

20. The pharmaceutical composition of claim 17, wherein the MTHFD inhibitor is a MTHFD1 inhibitor, MTHFD2 inhibitor, an MTHFD1L inhibitor or a combination thereof.

21. The pharmaceutical composition of claim 17, wherein at least one ferroptosis inducer is a glutathione peroxidase 4 (GPX4) inhibitor.

22. The pharmaceutical composition of claim 21, wherein the GPX4 inhibitor is RSL3, FIN56, FINO₂, ML-210, M-162, JKE-1674 or a combination thereof.

23. The pharmaceutical composition of claim 21, wherein a unit dose of the SHMT inhibitor when present is about 0.01 mg to about 1 g, a unit dose of the MTHFD inhibitor when present is about 0.01 mg to about 1 g, and a unit dose of the GPX4 inhibitor when present is about 0.01 mg to about 1 g.


SEQUENCE LISTING (10 sequences)

SEQ ID NO: 1 - RNA, 21 nt, synthetic construct
  source 1..21: mol_type = other RNA
ggcactaccagagctaactca

SEQ ID NO: 2 - RNA, 21 nt, synthetic construct
  source 1..21: mol_type = other RNA
gcatctcagagcgtcggttgc

SEQ ID NO: 3 - RNA, 21 nt, synthetic construct
  source 1..21: mol_type = other RNA
ggtgactgtgatctcgtacct

SEQ ID NO: 4 - RNA, 20 nt, synthetic construct
  source 1..20: mol_type = other RNA
gagaaggacaggcagtgtcg

SEQ ID NO: 5 - RNA, 20 nt, synthetic construct
  source 1..20: mol_type = other RNA
gtagacggccaggttggctg

SEQ ID NO: 6 - RNA, 21 nt, synthetic construct
  source 1..21: mol_type = other RNA
gagggagtggaacctcgatat

SEQ ID NO: 7 - RNA, 21 nt, synthetic construct
  source 1..21: mol_type = other RNA
gcgccaaccaggatcacactc

SEQ ID NO: 8 - RNA, 21 nt, synthetic construct
  source 1..21: mol_type = other RNA
gggtgaagcgctacggaccca

SEQ ID NO: 9 - RNA, 20 nt, synthetic construct
  source 1..20: mol_type = other RNA
atgctgggctcgcgagccgc

SEQ ID NO: 10 - RNA, 20 nt, synthetic construct
  source 1..20: mol_type = other RNA
tcccgattccaccgagacct